(19) United States
(12) Patent Application Publication
(10) Pub. No.: US 2016/0242672 A1
(43) Pub. Date: Aug. 25, 2016

(54) VITAL SIGNAL MEASURING APPARATUS AND METHOD FOR ESTIMATING CONTACT CONDITION

(71) Applicant: ASAHI KASEI KABUSHIKI KAISHA, Tokyo (JP)

(72) Inventors: Norihiko MIKOSHIBA, Tokyo (JP); Kei KODAMA, Tokyo (JP); Akihiro OKAMOTO, Tokyo (JP)

(73) Assignee: ASAHI KASEI KABUSHIKI KAISHA, Tokyo (JP)

(21) Appl. No.: 15/027,845

(22) PCT Filed: Oct. 10, 2014
(86) PCT No.: PCT/JP2014/005182

§ 371 Date: Apr. 7, 2016

(30) Foreign Application Priority Data
Oct. 17, 2013 (JP) 2013-216311

(51) Int. Cl. A61B 5/053; A61B 5/04; A61B 5/00; A61B 5/16

(52) U.S. Cl. CPC A61B 5/0533; A61B 5/165; A61B 5/04; A61B 5/681

(57) ABSTRACT

A vital signal measuring apparatus is attached to a living body to measure a vital signal of the living body, which includes an amplifier, an A/D converter, a processing unit, an n-th-order differential signal output unit, and an output unit. The amplifier and the A/D converter constitute a vital signal measuring unit for measuring a vital signal from a vital signal sensor and outputting a first signal corresponding to the vital signal. A time differentiation signal output unit includes the n-th-order differential signal output unit for receiving the first signal and outputting an n-th-order time differentiation signal (where n is an integer of 1 or more) of the first signal. The processing unit functions as a contact condition determination unit for determining contact condition between the vital signal measuring apparatus and the living body based on the n-th-order time differentiation signal.

### TECHNICAL FIELD

The present invention relates to a vital signal measuring apparatus and a method for estimating a contact condition, and more specifically to a vital signal measuring apparatus and a method for estimating a contact condition, which measures a vital signal such as skin conductivity or a pulse wave signal from a living body and also determines a contact condition between the vital signal measuring apparatus and the living body.

### BACKGROUND ART

Conventionally, there is an electrodermal activity (EDA) as a parameter indicating a mental activity state. This EDA is generally estimated to occur in such a manner that an electric phenomenon due to the activities of eccrine sweat glands is modified by the conditions of the epidermis, the ducts of sweat glands, and the like, which is deeply linked with a perspiration phenomenon. This electrodermal activity (EDA) is roughly divided into skin potential (skin potential activity; SPA) and skin conductance (skin conductance activity; SCA). The skin potential activity (SPA) is classified into skin potential level (SPL) and skin potential reflex (SPR).

The skin potential level (SPL) is a direct-current component of the skin potential activity (SPA). When the arousal level is high (in a moment of excitement), this SPL generally has a negatively high value, while when one feels sleepy or is in a relaxed state, the SPL trends in a positive direction.

The skin potential reflex (SPR) is an alternating-current component of the skin potential activity (SPA). The SPR frequently occurs by the stimulation of a change in external environment, such as a sensation of pain, a sense of touch, a sense of hearing, or a sense of vision, or by deep breathing or physical movement, or even when one does mental calculation or is lost in thought. The amplitude of the SPR is estimated to have an approximately-liner relationship with the intensity of a stimulus.

These activities are considered to reflect the function of a nervous activity of perspiration, and mental perspiration can be qualitatively known by observing this activity.

The electrodermal activity (EDA) is to represent electric properties resulting from a biochemical process and a physiological process in the skin surface or on the skin surface. This electrodermal activity (EDA) can be measured using a galvanometer, i.e., which operates by applying a fixed voltage between two regions on the skin surface and measuring current flowing between these regions to calculate electric conductance. A graph of this measurement with respect to time has two components.

In other words, there are a low-frequency tonic component (electrodermal level (also called “EDL”)) and a high-frequency phasic component (electrodermal response (also called “EDR”)) higher than that. The amplitude of the low-frequency component differs widely from person to person, and slowly varies with time as the skin adapts to environmental changes to achieve a constant state. The high frequency component is correlated with an individual psychophysical reaction to a stressful situation. Thus, the electrodermal activity (EDA) is considered to reflect a human psychological state, and applications to games and portable information terminals have been studied.

The electrodermal activity, such as a change in skin conductivity or skin potential, is known to change with a human emotional activity, which is one of vital signals measured by a so-called “lie detector.” Nowadays, there are cases where the applications of the electrodermal activity to the diagnosis of a mentally ill patient and the determination of the medical treatment progress are studied as well as the psychological applications, the electrodermal activity is incorporated in health equipment (wellness/healthcare equipment) as an indicator of health control or the amount of activity, and the like.

On the other hand, use of skin conductivity is widely applied for the purpose of training to control the stress of daily life. For example, the family of products, “Journey to Wild Divine” sold by United States Wild Divine, Inc., are to measure the skin conductivity and bloodstream pulse waves of a player of a video game to measure the heart rate from the bloodstream pulse waves in order to estimate a stress level from an emotional change of the player, which provide a mechanism for preventing the player from progressing to the next scene unless the player's emotional change is suppressed to conduct a mental exercise to suppress the emotional change (for example, see Non Patent Literature 1). As a result, the player can do the training of stress control.

Although the skin conductivity and other vital signals (vital signs) are used as biofeedback applications to the training to suppress the emotional change such as stress, this is often technology that focuses on detecting an unchanged state.

Further, for example, Patent Literature 1 relates to a device in which electrodes are provided in a teardrop-shaped housing to measure skin conductivity, which relates to a biofeedback method and device suitable for use in a biometric sensor, i.e., for stress control and entertainment applications. This sensor includes the housing having first and second surfaces, and these surfaces are electrodes suitable for detecting a biometric signal. This housing has the first surface, the second surface adapted to detect an electrodermal signal, and an element such as a processing element or a filter element. The element electrically communicates with the second surface, and is arranged within the housing. The element is configured to filter the electrodermal signal.

The technique disclosed in Patent Literature 1 is such that, among a sequence of measured values of skin conductivity continuously measured, an inclination is estimated by the least square method, for example, for every 16 pieces of data to increment or decrement an accumulator according to the inclination, and when exceeding a predetermined threshold value, the accumulator makes a transition to a predetermined stress state, thus continuously monitoring a user's anxiety level without the need for the extraction of specific events (for example, see paragraphs [0068] and [0069] of Patent Literature 1).

Further, for example, Patent Literature 2 relates to an emotion recognition system for monitoring a vital signal of electrodermal activity (EDA) to recognize a user's emotional state.

Further, for example, Patent Literature 3 discloses a device for performing frequency analysis or the like from bioinformation time-series data to calculate a feature amount, and comparing the calculated feature amount with a reference value as a normal feature amount to calculate whether noise is mixed in.

### CITATION LIST

### Patent Literatures

PTL 1: JP 2010-518914 A
PTL 2: JP 2004-474 A
PTL 3: JP H07-059738 A

### Non Patent Literature

NPL 1: http://www.wilddivine.com/complete-programs/journey-to-wild-divine-the-passage-complete-with-iom/Summary of Invention

### Technical Problem

Both of the techniques disclosed in Patent Literatures 1 and 2 mentioned above are vital signal measuring apparatuses for measuring a vital signal in contact with a living body to estimate a state of the living body such as emotion from the measured vital signal.

However, such a vital signal measuring apparatus to measure a vital signal in contact with a living body may change a contact condition between the living body and the vital signal measuring apparatus due to the movement of the living body. When the contact condition changes, the measurement accuracy of the vital signal is reduced. In other words, the signal measured by the vital signal measuring apparatus becomes a signal having a low percentage of the vital signal with noise or an artifact mixed in. When the contact condition changes in this way, an error in estimating the state of the living body such as emotion occurs. To reduce the estimation error or remove the noise or artifact from the measured signal, it is necessary to detect the fact that the contact condition has changed.

Further, the technique disclosed in Patent Literature 3 mentioned above is to calculate a feature amount and calculate a difference from a stored reference feature amount in order to determine a contact failure. This not only increases the amount of computation, but has a problem that the change in contact condition with the living body cannot be determined accurately in real time.

The present invention has been made in view of such a problem, and it is an object thereof to provide a vital signal measuring apparatus and a method for estimating a contact condition, capable of determining a change in contact condition with a living body accurately in real time.

### Solution to Problem

According to one aspect of the present invention, the following is characterized:

(1) A vital signal measuring apparatus attached to a living body to measure a vital signal of the living body, including: a vital signal measuring unit for measuring the vital signal and outputting a first signal corresponding to the vital signal; a time differentiation signal output unit for receiving the first signal, and outputting an n-th-order time differentiation signal (where n is an integer of 1 or more) of the first signal; and a contact condition determination unit for determining a contact condition between the vital signal measuring apparatus and the living body based on the n-th-order time differentiation signal.

(2) In (1), an emotional change estimating unit for estimating an emotional change of the living body based on the n-th-order time differentiation signal is further included.

(3) In (2), the emotional change estimating unit compares the n-th-order time differentiation signal with a first threshold value to estimate the emotional change of the living body.

(4) In (2) or (3) the contact failure determination unit determines that the contact condition between the vital signal measuring apparatus and the living body has changed based on the n-th-order time differentiation signal and a negative threshold value.

(5) In any one of (2) to (4), when a change in which the n-th-order time differentiation signal is lower than a negative second threshold value and higher than a positive third threshold value is repeated as many times as a value obtained by applying a floor function (n+1)/2, the contact condition determination unit determines that the contact condition between the vital signal measuring apparatus and the living body has changed.

(6) In any one of (2) to (4), when a change in which the n-th-order time differentiation signal is higher than a positive fourth threshold value and lower than a negative fifth threshold value is repeated as many times as a value obtained by applying a floor function (n+1)/2, the contact condition determination unit determines that the contact condition between the vital signal measuring apparatus and the living body has changed.

(7) In anyone of (2) to (6), when the contact condition determination unit determines that the contact condition has changed, the emotional change estimating unit corrects data on the n-th-order time differentiation signal at a corresponding first period, generates a second signal, and estimates the emotional change based on the second signal.

(8) In (7), the emotional change estimating unit corrects the data on the n-th-order time differentiation signal at the first period based on the n-th-order time differentiation signal at a period other than the first period.

(9) In (3), the emotional change estimating unit changes the first threshold value according to the first signal.

(10) In (9), the emotional change estimating unit changes the first threshold value so that the first threshold value shall be a monotonic function of an average value of the first signal.

(11) In (1), the contact condition determination unit determines that the contact condition between the vital signal measuring apparatus and the living body has changed based on the n-th-order time differentiation signal and a negative threshold value.

(12) In (1) or (11), when a change in which the n-th-order time differentiation signal is lower than a negative second threshold value and higher than a positive third threshold value is repeated as many times as a value obtained by applying a floor function (n+1)/2, the contact condition determination unit determines that the contact condition between the vital signal measuring apparatus and the living body has changed.

(13) In (1) or (11), when a change in which the n-th-order time differentiation signal is higher than a positive fourth threshold value and lower than a negative fifth threshold value is repeated as many times as a value obtained by applying a floor function (n+1)/2, the contact condition determination unit determines that the contact condition between the vital signal measuring apparatus and the living body has changed.

(14) In anyone of (11) to (13), a first data correction unit for correcting data on the first signal at a corresponding first period to generate a second signal when the contact condition determination unit determines that the contact condition has changed is further included.

(15) In (14), the first data correction unit corrects the data on the first signal at the first period based on the first signal at a period other than the first period.

(16) In (1), (12), or (13), the time differentiation signal output unit outputs an m-th-order time differentiation signal (where m is an integer larger than n) of the first signal, and the vital signal measuring apparatus further includes a noise signal determination unit for determining whether the first signal contains a noise signal caused by a change in contact condition between the vital signal measuring apparatus and the living body based on the n-th-order time differentiation signal and the m-th-order time differentiation signal.

(17) In (16), when the ratio of the amplitude of the m-th-order time differentiation signal to the amplitude of the n-th-order time differentiation signal is larger than a sixth threshold value, the noise signal determination unit determines that the noise signal is contained, and when the ratio of the amplitude of the m-th-order time differentiation signal to the amplitude of the n-th-order time differentiation signal is smaller than a seventh threshold value, the noise signal determination unit determines that the noise signal is not contained.

(18) In (16) or (17), the contact condition determination unit further includes a second data correction unit for correcting data on the first signal at a corresponding second period and generating a third signal when the noise signal determination unit determines that the noise signal is contained.

(19) In (18), the second data correction unit corrects the data on the first signal at the second period based on the first signal at a period other than the second period.

(20) In (16) or (17), an emotional change estimating unit for estimating an emotional change of the living body based on the n-th-order time differentiation signal is further included, wherein the emotional change estimating unit corrects data on the n-th-order time differentiation signal at a corresponding second period, generates a third signal, and estimates the emotional change based on the third signal when the noise signal determination unit determines that the noise signal is contained.

(21) In (20), the emotional change estimating unit corrects the data on the n-th-order time differentiation signal at the second period based on the n-th-order time differentiation signal at a period other than the second period.

(22) In any one of (1) to (21), the first signal and the n-th-order time differentiation signal are discrete time signals.

(23) In any one of (1) to (22), the vital signal measuring unit is an electrophysical quantity measuring unit for measuring an electrophysical quantity on at least one electrode adapted to come into contact with the skin of the living body, and outputting a signal corresponding to the electrophysical quantity as the first signal.

(24) In any one of (1) to (22), the vital signal measuring unit measures a pulse wave of the living body from a plethysmogram sensor, and outputs a signal corresponding to the pulse wave as the first signal.

(25) A method for estimating a contact condition, which determines a contact condition of a vital signal measuring apparatus for measuring a vital signal in contact with a living body, including: measuring the vital signal and outputting a first signal corresponding to the vital signal; outputting an n-th-order time differentiation signal (where n is an integer of 1 or more) of the first signal; and determining a contact condition between the vital signal measuring apparatus and the living body based on the n-th-order time differentiation signal.

(26) A program for causing a computer to execute the method for estimating a contact condition according to (25).

(27) A computer-readable recording medium on which the program according to (26) is recorded.

### Advantageous Effect of Invention

According to one aspect of the present invention, a change in contact condition with a living body can be determined accurately in real time.

### BRIEF DESCRIPTION OF DRAWINGS

FIG. 1 is a block diagram for describing an embodiment of a vital signal measuring apparatus according to the present invention;

FIG. 2 is a block diagram for describing Embodiment 1 of an emotional change estimating apparatus according to the present invention;

FIG. 3 is a chart illustrating a skin conductive value obtained from an electrophysical quantity measuring unit illustrated in FIG. 2;

FIGS. 4A and 4B are waveform charts of skin conductivity, a first-order differential signal, and the output of an emotional change estimating unit;

FIG. 5 is a block diagram for describing Embodiment 2 of an emotional change estimating apparatus according to the present invention;

FIGS. 6A to 6C are waveform charts of outputs of the skin conductivity, the first-order differential signal, and a second-order differential signal when a contact condition between a skin and an electrode becomes instantaneously failure;

FIGS. 7A to 7C are waveform charts of outputs of the skin conductivity, the first-order differential signal, and the second-order differential signal when the contact condition between the skin and the electrode changes;

FIGS. 8A to 8C are waveform charts of outputs of the skin conductivity, the first-order differential signal, and the second-order differential signal when the contact condition between the skin and the electrode repeats a contact failure with a small amplitude;

FIGS. 9A to 9D are waveform charts of outputs of the first-order differential signal and the second-order differential signal before and after the correction of a contact failure portion between the skin and the electrode;

FIGS. 10A and 10B are waveform charts of the skin conductivity, the first-order differential signal, and the output of the emotional change estimating unit when the skin conductivity increases;

FIG. 11 is a block diagram for describing Embodiment 5 of an emotional change estimating apparatus according to the present invention;

FIGS. 12A to 12C are charts for comparing amplitude differences between a first-order difference and a second-order difference with and without a contact failure;

FIG. 13 is a block diagram for describing Embodiment 6 of an emotional change estimating apparatus according to the present invention;

FIG. 14 is a block diagram for describing Embodiment 7 of an emotional change estimating apparatus according to the present invention;

FIG. 15 is a flowchart for describing an emotional change estimating method according to the present invention;

FIG. 16 is another flowchart for describing another emotional change estimating method according to the present invention;

FIG. 17 is a block diagram of a plethysmogram measuring apparatus including a light-emitting source and a light-receiving element, where light obtained by transmitting or reflecting light from a light-emitting element through or on a living body is received by the light-receiving element to obtain a pulsation signal from output of the light-receiving element;

FIGS. 18A to 18C are measured waveform charts of an example of a plethysmogram signal including an artifact signal caused by a contact failure, and also illustrating the first-order differential signal and the second-order differential signal; and

FIGS. 19A and 19B are diagrams illustrating a specific example of a sensor unit for realizing each of the aforementioned embodiments.

### DESCRIPTION OF EMBODIMENTS

In the following detailed description, many specific details are described to provide thorough understanding of embodiments of the present invention. However, it will be obvious that one or more embodiments can be carried out without such specific details. In addition, known structures and devices are illustrated in schematic diagrams to simplify the drawings.

Each embodiment of a vital signal measuring apparatus and a method for estimating a contact condition of the present invention will be described below with reference to the accompanying drawings. In the following description of each embodiment, an emotional change estimating apparatus will be described as an example of the vital signal measuring apparatus, and an emotional change estimating method will be described as an example of the method for estimating a contact condition. In other words, the present invention is not limited to the emotional change estimating apparatus and the emotional change estimating method.

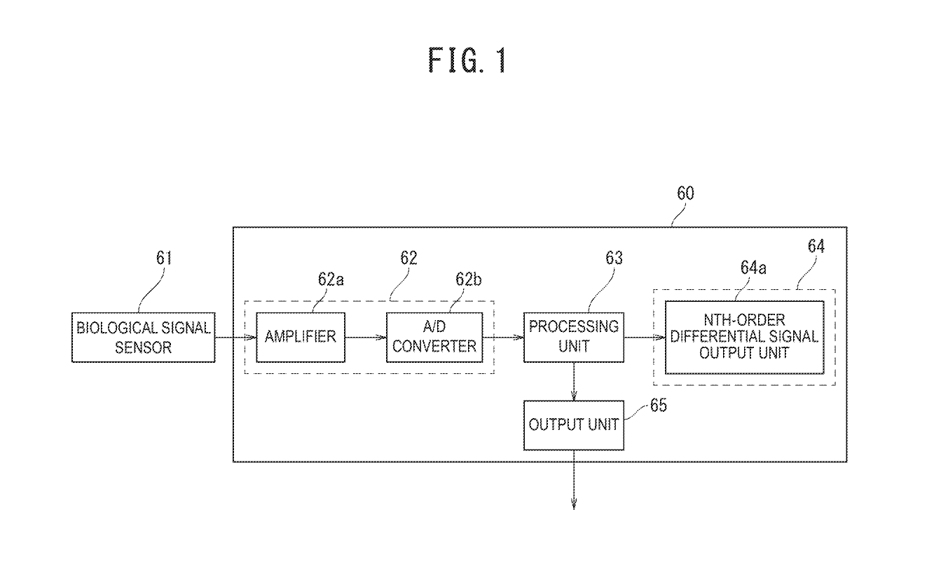

FIG. 1 is a block diagram for describing an embodiment of the vital signal measuring apparatus according to the present invention. In the figure, y 60 denotes a vital signal measuring apparatus, 62 is a vital signal measuring unit, 62a is an amplifier, 62b is an A/D converter, 63 is a processing unit, 64 is a time differentiation signal output unit, 64a is an n-th-order differential signal output unit, and 65 is an output unit.

The vital signal measuring apparatus 60 of the embodiment is a vital signal measuring apparatus attached to a living body to measure a vital signal of the living body, including the amplifier 62a, the A/D converter 62b, the processing unit 63, the n-th-order differential signal output unit 64a, and the output unit 65. The amplifier 62a and the A/D converter 62b constitute a vital signal measuring unit 62 for measuring a vital signal from a vital signal sensor 61 to output a first signal corresponding to the vital signal.

The time differentiation signal output unit 64 includes an n-th-order differential signal output unit 64a for receiving the first signal and outputting an n-th-order time differentiation signal (where n is an integer of 1 or more) of the first signal.

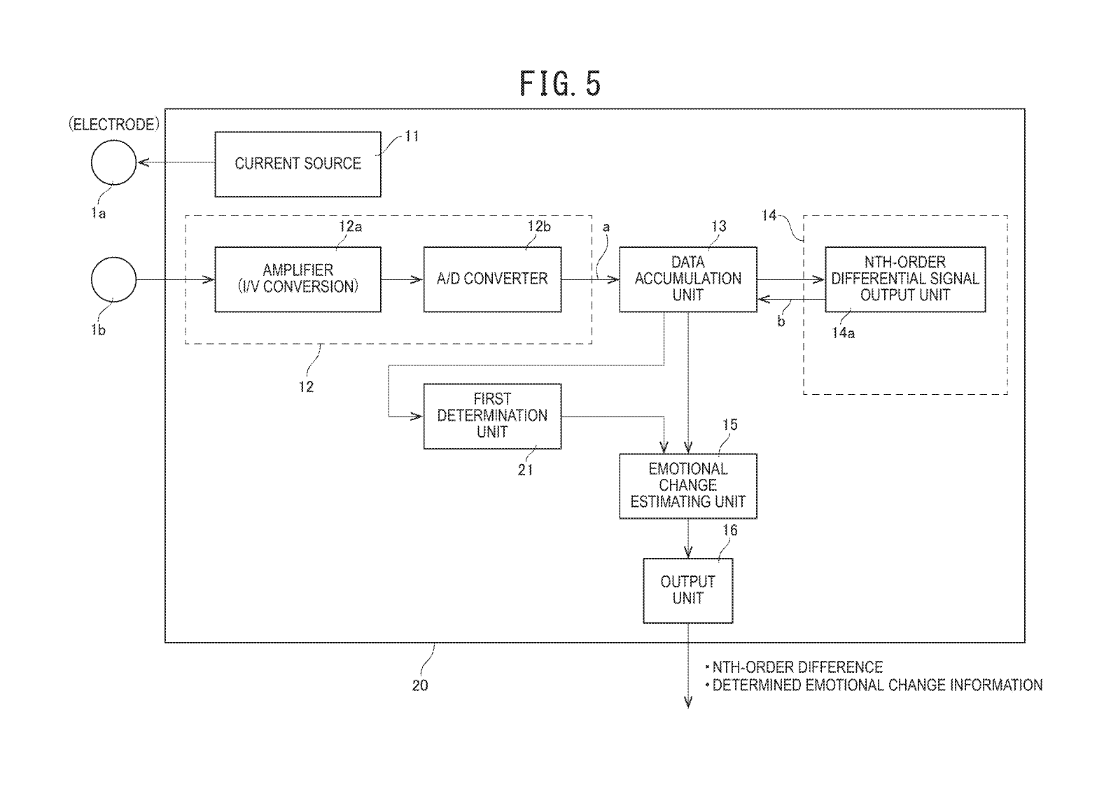

The processing unit 63 functions as a contact condition determination unit (a first determination unit 21 in FIG. 5) to determine a contact condition between the vital signal measuring apparatus 60 and a living body based on the n-th-order time differentiation signal.

An emotional change estimating unit (denoted by reference symbol 15 in FIG. 5) to estimate an emotional change of the living body based on the n-th-order time differentiation signal is further included. The emotional change estimating unit 15 compares the n-th-order time differentiation signal with a first threshold value to estimate the emotional change of the living body.

Based on the n-th-order time differentiation signal and a negative threshold value, the contact condition determination unit 21 determines that the contact condition between the vital signal measuring apparatus 60 and the living body has changed.

When a change in which the n-th-order time differentiation signal is lower than a negative second threshold value and higher than a positive third threshold value is repeated as many times as a value obtained by applying a floor function (n+1)/2, the contact condition determination unit 21 determines that the contact condition between the vital signal measuring apparatus 60 and the living body has changed.

Further, when a change in which the n-th-order time differentiation signal is higher than a positive fourth threshold value and lower than a negative fifth threshold value is repeated as many times as a value obtained by applying the floor function (n+1)/2, the contact condition determination unit 21 determines that the contact condition between the vital signal measuring apparatus 60 and the living body has changed.

When the contact condition determination unit 21 determines that the contact condition has changed, the emotional change estimating unit 15 corrects data on the n-th-order time differentiation signal at a corresponding first period to generate a second signal in order to estimate an emotional change based on the second signal.

Further, the emotional change estimating unit 15 corrects data on the n-th-order time differentiation signal at the first period based on the n-th-order time differentiation signal at a period other than the first period.

The emotional change estimating unit 15 changes the first threshold value according to the first signal. Further, the emotional change estimating unit 15 changes the first threshold value so that the first threshold value shall be a monotonic function of an average value of the first signal.

Further, the contact condition determination unit 21 determines that the contact condition between the vital signal measuring apparatus 60 and the living body has changed based on the n-th-order time differentiation signal and the negative threshold value.

Further, when a change in which the n-th-order time differentiation signal is lower than the negative second threshold value and higher than the positive third threshold value is repeated as many times as a value obtained by applying the floor function (n+1)/2, the contact condition determination unit 21 determines that the contact condition between the vital signal measuring apparatus 60 and the living body has changed.

Further, when a change in which the n-th-order time differentiation signal is higher than the positive fourth threshold value and lower than the negative fifth threshold value is repeated as many times as a value obtained by applying the floor function (n+1)/2, the contact condition determination unit 21 determines that the contact condition between the vital signal measuring apparatus 60 and the living body has changed.

The contact condition determination unit 21 further has a first data correction unit 63, 15 for correcting data on the first signal at a corresponding first period to generate a second signal when the contact condition determination unit 21 determines that the contact condition has changed.

The first data correction unit 63, 15 corrects the data on the first signal at the first period based on the first signal at a period other than the first period.

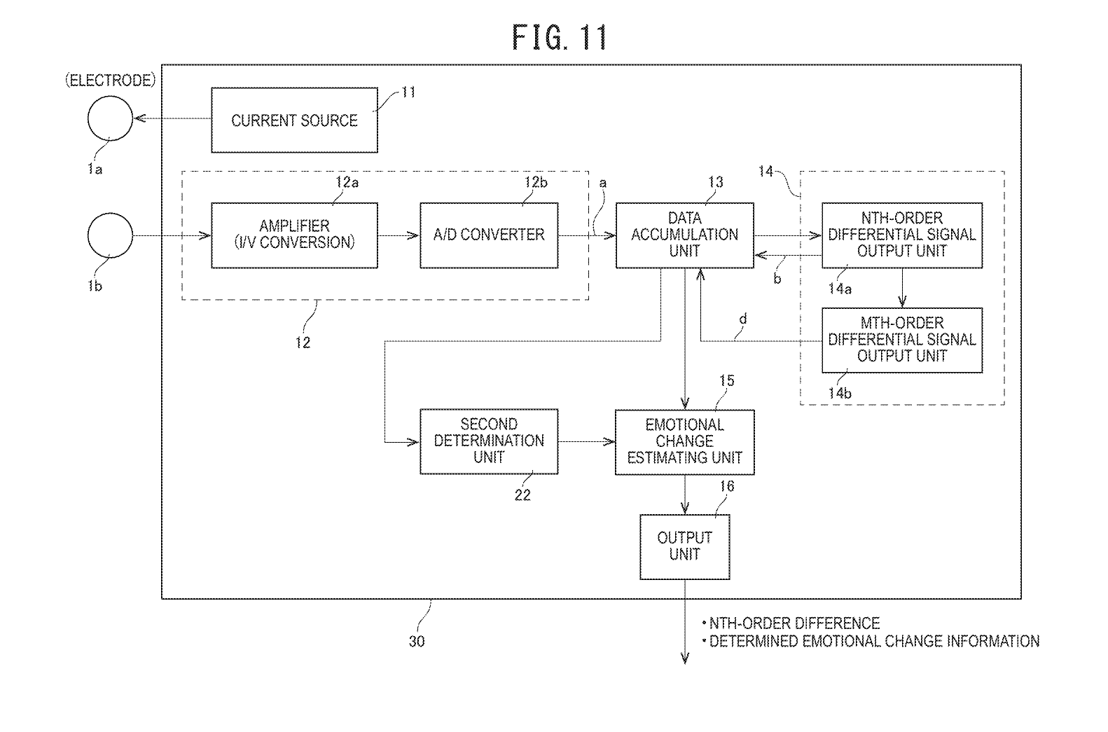

The time differentiation signal output unit 64 further includes a noise signal determination unit (a second determination unit in FIG. 11) 22 for outputting an m-th-order time differentiation signal (where m is an integer larger than n) of the first signal to determine whether the first signal contains a noise signal caused by a change in contact condition between the vital signal measuring apparatus 60 and the living body based on the n-th-order time differentiation signal and the m-th-order time differentiation signal.

When the ratio of the amplitude of the m-th-order time differentiation signal to the amplitude of the n-th-order time differentiation signal is larger than a sixth threshold value, the noise signal determination unit 22 determines that the noise signal is contained, whereas when the ratio of the amplitude of the m-th-order time differentiation signal to the amplitude of the n-th-order time differentiation signal is smaller than a seventh threshold value, the noise signal determination unit 22 determines that no noise signal is contained.

The contact condition determination unit 21 further has a second data correction unit (an emotional change estimating unit in FIG. 5) 15 for correcting data on the first signal at a corresponding second period to generate a third signal when the noise signal determination unit 22 determines that the noise signal is contained.

The second data correction unit 15 corrects the data on the first signal at the second period based on data on the first signal at a period other than the second period.

The emotional change estimating unit 15 for estimating an emotional change of the living body based on the n-th-order time differentiation signal is further included. When the noise signal determination unit 22 determines that the noise signal is contained, the emotional change estimating unit 15 corrects data on the n-th-order time differentiation signal at a corresponding second period to generate a third signal in order to estimate the emotional change based on the third signal.

The emotional change estimating unit 15 corrects the data on the n-th-order time differentiation signal at the second period based on data on the n-th-order time differentiation signal at a period other than the second period.

The first signal and the n-th-order time differentiation signal are discrete time signals.

The vital signal measuring unit 62 is an electrophysical quantity measuring unit for measuring an electrophysical quantity on at least one electrode adapted to come into contact with the skin of the living body, and outputting a signal corresponding to the electrophysical quantity as the first signal.

Further, the vital signal measuring unit 62 measures pulse waves of the living body from plethysmogram sensors 71a, 71b, and outputs a signal corresponding to the pulse waves as the first signal.

The method for estimating a contact condition according to the present invention is a contact condition estimating method for determining a contact condition of the vital signal measuring apparatus 60 for measuring a vital signal in contact with a living body.

The method includes: measuring a vital signal and outputting a first signal corresponding to the vital signal; outputting an n-th-order time differentiation signal (where n is an integer of 1 or more) of the first signal; and determining a contact condition between the vital signal measuring apparatus 60 and the living body based on the n-th-order time differentiation signal.

There is also provided a program for causing a computer to execute the above-mentioned contact condition estimating method. Further, there is provided a computer-readable recording medium with the program recorded thereon.

### Embodiment 1

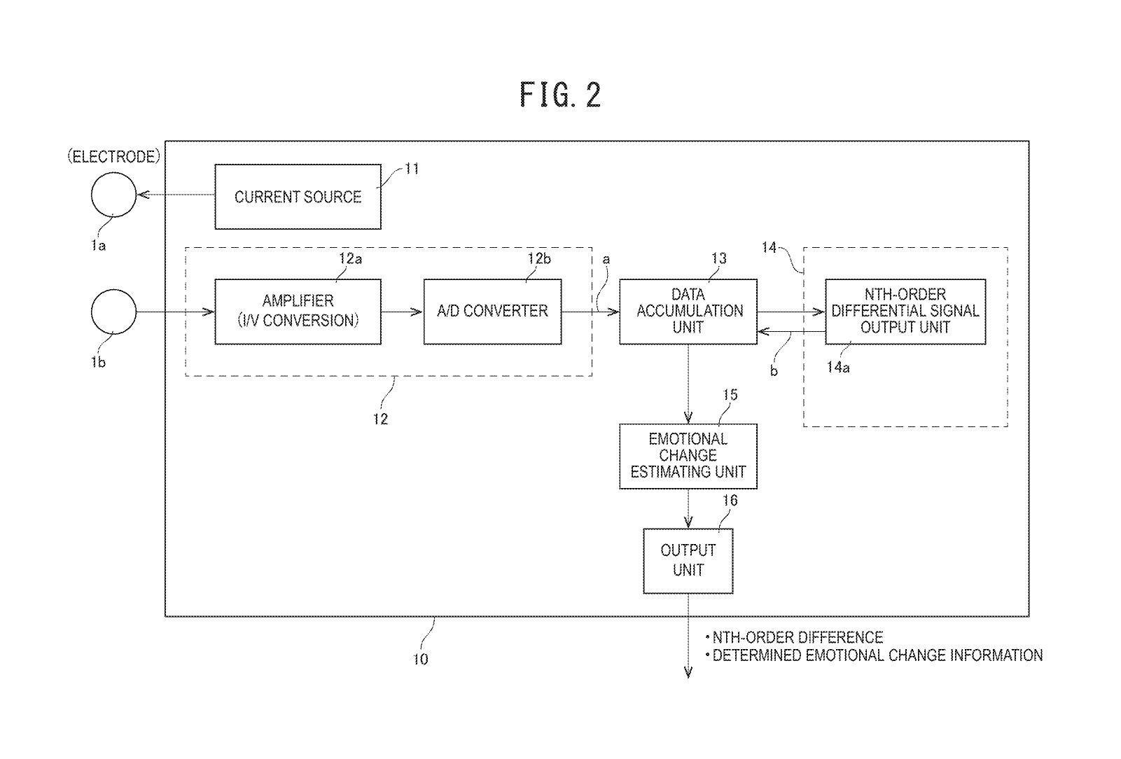

FIG. 2 is a block diagram for describing Embodiment 1 of an emotional change estimating apparatus according to the present invention. In the figure, reference symbols 1a and 1b denote electrodes, 10 is an emotional change estimating apparatus, 11 is a current source, 12 is an electrophysical quantity measuring unit, 12a is an amplifier (I/V conversion), 12b is an A/D converter, 13 is a data accumulation unit, 14 is a differential signal output unit, 14a is an n-th-order differential signal output unit, 15 is an emotional change estimating unit, and 16 is an output unit.

The emotional change estimating apparatus 10 of Embodiment 1 is an emotional change estimating apparatus for measuring the electrodermal activity of a living body to estimate a change in excited state of this living body, including the electrophysical quantity measuring unit 12, the differential signal output unit 14, and the emotional change estimating unit 15. The emotional change estimating apparatus 10 is incorporated in wristband type electronic equipment or the like worn on the living body.

The electrophysical quantity measuring unit 12 measures an electrophysical quantity on at least one of the electrodes 1a and 1b adapted to come into contact with the skin of the living body, and outputs a first signal a corresponding to this electrophysical quantity, including the amplifier (I/V conversion) 12a and the A/D converter 12b.

The electrophysical quantity measuring unit 12 is a vital signal measuring unit for measuring a vital signal from the living body, and outputting a first signal a corresponding to the vital signal.

The differential signal output unit 14 takes in input of data on the first signal a at multiple times to output an n-th-order differential signal (where n is an integer b of one or more) of the first signal a, including the n-th-order differential signal output unit 14a. Here, the n-th-order differential signal is an n-th-order change-rate signal of the first signal a. The n-th-order differential signal output unit 14a is a time differentiation signal output unit for receiving the first signal a, and outputting an n-th-order time differentiation signal of the first signal a.

The first signal a and the n-th-order differential signal b are discrete time signals. Further, the n-th-order differential signal b is a signal obtained from data adjacent in terms of time in the first signal a. The integer in the n-th-order differential signal b is n=1.

Although Embodiment 1 is to perform discrete time signal processing, continuous-time signal processing may be performed without providing the A/D converter. In this case, the n-th-order change-rate signal is an n-th-order differential signal, and the time differentiation signal output unit is an n-th-order differential signal output unit. In each of the following embodiments, description will be made of a configuration for performing discrete time signal processing, but the configuration may perform continuous-time signal processing.

The emotional change estimating unit 15 estimates an emotional change of the living body based on the n-th-order differential signal b from the differential signal output unit 14.

In Embodiment 1, the two electrodes 1a and 1b are adapted to come into contact with the skin of the living body, where current is supplied from the current source to one electrode 1a. When the two electrodes 1a and 1b are in contact with the skin of the living body, this current flows from one electrode 1a to the other electrode 1b through the skin of the living body. Then, the electrophysical quantity measuring unit 12 measures an electrophysical quantity at the other electrode 1b. Note that the electrophysical quantity means an electrically physical quantity such as the electric conductivity, the resistance value, the capacitance value, or the skin potential.

In Embodiment 1, the electrophysical quantity measuring unit 12 I/V converts the current flowing between the two electrodes 1a and 1b, which is composed of the amplifier 12 for measuring voltage at the other electrode 1b and the A/D converter 12b for A/D converting the measured voltage to output a digital signal as a corresponding discrete time signal. Here, when the two electrodes 1a and 1b are in contact with the skin of the living body, the digital signal output from the A/D converter 12b is a signal corresponding to the electric conductivity of the living body.

The blocks downstream of the A/D converter 12b can be realized by a logic circuit or by a processor for loading a program to carry out predetermined operations, such as a DSP (digital signal processor; microprocessor dedicated to process sound and images) or a CPU.

The digital signal output from the A/D converter is input to the data accumulation unit 13, and then input to the differential signal output unit 14 after being buffered. The data accumulation unit 13 can be realized by a known memory circuit, such as a register, a RAM or a cache memory. Note that the data accumulation unit 13 of Embodiment 1 is unnecessary as long as the A/D converter 12b or the differential signal output unit 14 has a data accumulation function, namely a buffer function.

The differential signal output unit 14 takes in input of data on the digital signal accumulated in the data accumulation unit 13 at multiple times to output an n-th-order differential signal (where n is an integer of 1 or more) of the digital signal in the n-th-order differential signal output unit 14a. In Embodiment 1, the n-th-order differential signal is a first-order differential signal, namely a signal obtained from data adjacent in terms of time. In the following figures, each discrete time signal is plotted as a continuous graph, rather than as a point graph or a bar graph, by connecting vertexes of respective point or bar graphs for ease of comprehension. The first-order differential signal is buffered in the data accumulation unit 13, and then input to the emotional change estimating unit 15.

Based on the first-order differential signal, the emotional change estimating unit 15 estimates an emotional change of the living body. Specifically, the emotional change estimating unit 15 compares the first-order differential signal with a threshold value to estimate the emotional change.

Then, the emotional change estimating unit 15 outputs the presence or absence of the emotional change and the degree of change in the emotional change as the estimation results to the outside through the output unit 16. Note that the output unit 16 is not an indispensable constituent feature of the present invention. The output unit 16 is made up of a buffer, a display, and the like. When the output unit 16 is configured with a display, the emotional change estimating apparatus 15 can inform a user of emotional change information.

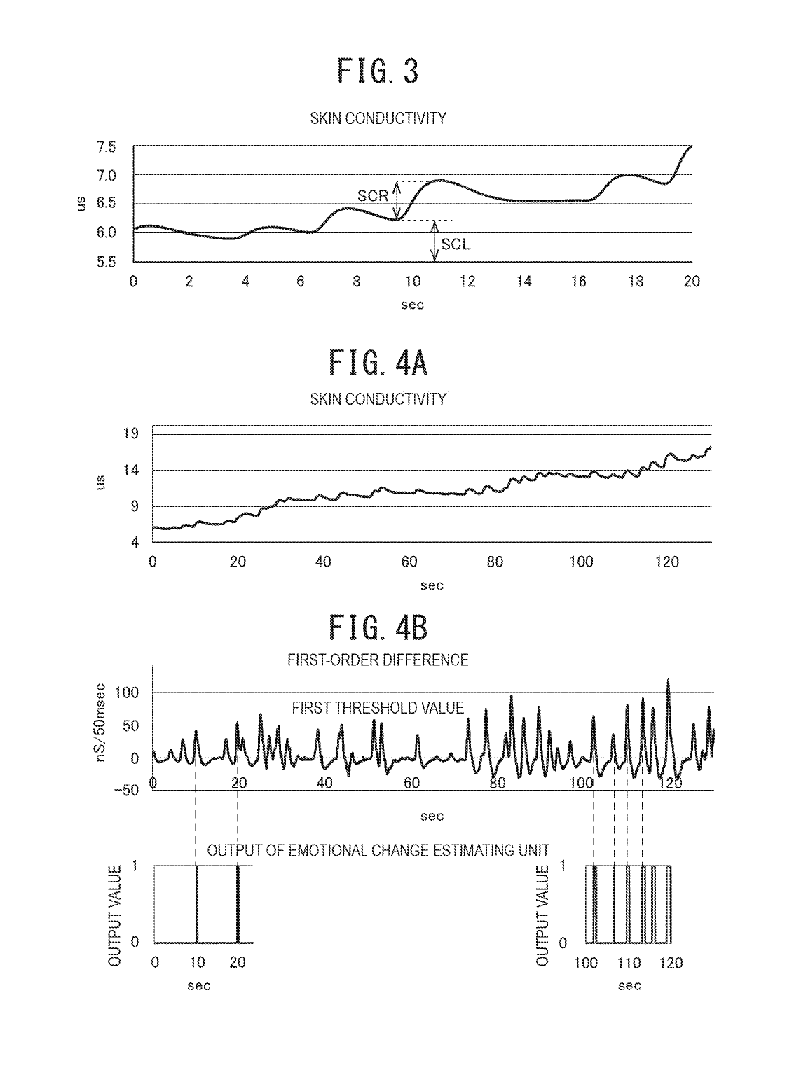

FIG. 3 is a chart illustrating a skin conductive value obtained from the electrophysical quantity measuring unit illustrated in FIG. 2. The skin conductive value (μs: microsiemens) is made up of a slow change and an abrupt change. The abrupt change is called a skin conductive reaction (SCR) or a phase component (Phasic), and the basal value of the slow change is called a skin conductive level (SCL) or a tonic component (Tonic).

The SCR means that the measured value of skin conductivity between electrodes increases under the influence of the electric conductivity of the dermis, rather than the epidermis, due to the opening of sweat glands in the skin of the human body, and it is known that the reaction of SCR is seen accompanied by a human emotional change. On the other hand, it is said that the SCL has relevance to the perspiration situation of the skin surface, the overall mental exaltation situation, and the like.

A momentary emotional change of a human can be represented as an SCR change value. However, since the measured value of skin conductivity is observed in such a manner that the SCR waveform is superimposed on the SCL value as illustrated in FIG. 3, the SCR value cannot be extracted based merely on the magnitude of the value of skin conductivity.

In the present invention, a differential value of measured values of skin conductivity, adjacent in terms of time, is computed to evaluate the differential value as the SCR. This can lead to extracting only the reaction component of the SCR without evaluating the SCL basal level.

FIGS. 4A and 4B are waveform charts of the skin conductivity, the first-order differential signal, and the output of the emotional change estimating unit, where FIG. 4A is a chart illustrating the skin conductivity and FIG. 4B is a chart illustrating the first-order differential signal and the output of the emotional change estimating unit.

The emotional change estimating unit 15 compares the n-th-order differential signal b and the first threshold value to estimate an emotional change of the living body. In other words, the first-order differential signal is compared with a given threshold value in the emotional change estimating unit 15. Then, when the first-order differential signal is larger than the threshold value, 1 is output, while when the first-order differential signal is smaller than the threshold value, 0 is output. 1 indicates that there is a large emotional change, and 0 indicates that the emotional change is small or there is no emotional change.

According to the above configuration and operation, since the emotional change estimating apparatus 10 of Embodiment 1 can detect a change in electrophysical quantity such as the skin conductivity of the living body at high speed, the emotional change of the living body can be estimated at high speed.

Although the first-order differential value of the above measured values of skin conductivity is mentioned, the SCR reaction value can be extracted by separating from the SCL even if a second-order differential value is determined and evaluated. One concept of the present invention is that an n-th-order difference of skin conductivity (where n is a natural number) is evaluated to extract a value corresponding to the SCR.

Information indicating that there is an SCR reaction is determined by the emotional change estimating unit 15, and output to the outside from the output unit. The output SCR value can be operated as an index indicating that there is an emotional change (of a test subject), and used in a psychometric test, as an index of an activity level, or as reaction to a visual or acoustic stimulus typified by a game, and applied to the purpose of biofeedback and the like.

Further, a difference between adjacent measured values in continuous measured values of skin conductivity may be derived to evaluate the differential value with a predetermined threshold value, estimate the emotional state of the subject being tested, and output the evaluated value associated with the emotional state, or evaluate the differential value with two or more threshold values and output a differential value as an evaluated value weighted for each threshold value.

### Embodiment 2

FIG. 5 is a block diagram for describing Embodiment 2 of an emotional change estimating apparatus according to the present invention. In the figure, 20 denotes an emotional change estimating apparatus, and 21 is a first determination unit. Note that constituent features having the same functions as those in FIG. 2 are given the same reference symbols.

The emotional change estimating apparatus 20 of Embodiment 2 is an emotional change estimating apparatus for measuring the electrodermal activity of a living body to estimate a change in excited state of this living body, including an electrophysical quantity measuring unit 12, a differential signal output unit 14, an emotional change estimating unit 15, and the first determination unit 21.

The first determination unit 21 determines whether a contact condition between the skin and the electrode 1a, 1b based on the n-th-order differential signal b. In other words, the emotional change estimating apparatus 20 of Embodiment 2 further includes the first determination unit 21 in the emotional change estimating apparatus 10 of Embodiment 1 mentioned above to determine whether the contact condition between the skin and the electrode has changed based on the n-th-order differential signal, i.e., the first determination unit 21 is a contact condition determination unit for determining the contact condition between the vital signal measuring apparatus and the living body based on the n-th-order time differentiation signal.

The first determination unit 21 takes in input of a first-order differential signal held in the data accumulation unit 13, and makes comparison with a negative second threshold value and a positive third threshold value. Then, when the first-order differential signal has changed to be lower than the negative second threshold value and higher than the positive third threshold value, the first determination unit 21 determines that the contact condition between the skin and the electrode has changed.

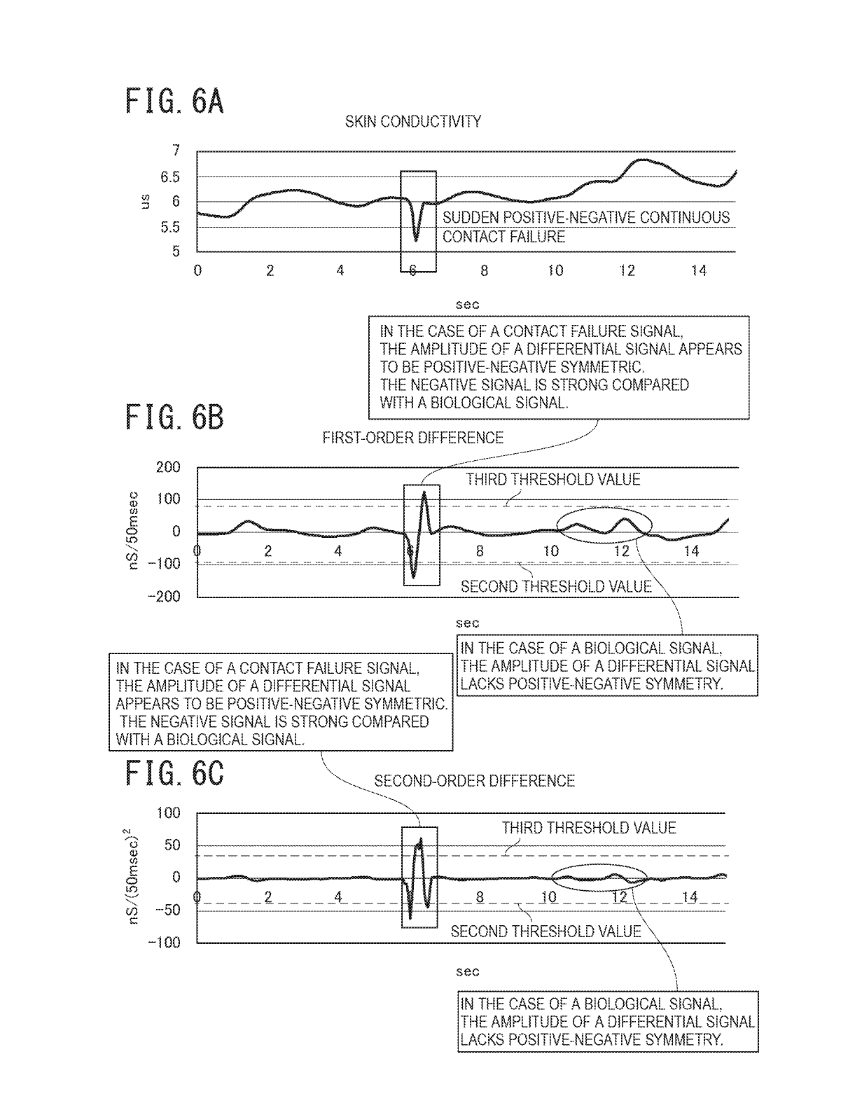

FIGS. 6A to 6C are waveform charts of outputs of the skin conductivity, the first-order differential signal, and a second-order differential signal when the contact condition between the skin and the electrode becomes instantaneously failure, where FIG. 6A is a chart illustrating the skin conductivity, FIG. 6B is a chart illustrating the first-order differential signal and the output of the emotional change estimating unit, and FIG. 6C is a chart illustrating the second-order differential signal and the output of the emotional change estimating unit. The waveforms of the skin conductivity, the first-order differential signal, and the second-order differential signal when the contact condition between the skin and the electrode becomes instantaneously failure are illustrated.

When a change in which the n-th-order differential signal b is lower than the negative second threshold value and higher than the positive third threshold value is repeated as many times as a value obtained by applying the floor function (n+1)/2, the first determination unit 21 in the emotional change estimating apparatus 20 of Embodiment 2 determines that the contact condition between the skin and the electrode 1a, 1b has changed. Then, the first determination unit 21 outputs this determination result to the emotional change estimating unit 15.

For example, such an event that the contact between the electrode and the skin becomes instantaneously failure often occurs when a skin conductivity measuring apparatus (emotional change estimating apparatus) has a wristwatch type shape and an arm is moved during walking while attaching the apparatus to the wrist with a band or the like. In such an occasion, the measured value of skin conductivity is observed with abrupt noise superimposed as illustrated in FIG. 6A. The result of performing first-order difference processing on this measured value to extract an SCR reaction is illustrated in FIG. 6B. As seen from this result, noise associated with a contact failure between the electrode and the skin is observed as a value larger than the SCR reaction observed therearound in the first-order differential value.

When a sudden contact failure occurs between the skin and the electrode, electric conductivity between the electrodes becomes low. When the electric conductivity between the electrodes becomes low, the first-order differential signal changes to a negative value and to a positive value. In Embodiment 2, when the first-order differential signal becomes lower than the negative second threshold value and higher than the positive third threshold value, a signal indicating that the contact condition has changed is output from the first determination unit 21 to the emotional change estimating unit 15. For example, the signal indicating contact condition change is 0 when there is no change and 1 when there is a change.

As illustrated in FIG. 6C, the second-order differential signal can be used to determine whether the contact condition has changed. When a sudden contact failure occurs between the skin and the electrode, the second-order differential signal changes in the order of a negative value, a positive value, and a negative value.

In other words, even if the second-order differential signal is used, the contact condition change between the skin and the electrode can be determined when the second-order differential signal has changed to be lower than the negative second threshold value and higher than the positive third threshold value. Note that a change in the order that the second-order differential signal becomes lower than the negative second threshold value, becomes higher than the positive third threshold value, and becomes lower than the negative second threshold value again may be detected to determine a change in contact condition.

Similarly, in the case of using a third-order differential signal, though not illustrated, when there is a sudden change in contact condition, the third-order differential signal changes in this order of a negative value, a positive value, a negative value, and a positive value. When an n-th-order differential signal is used, a change of the n-th-order differential signal to a negative value and a positive value is repeated as many times as floor{(n+1)/2}. Further, when n is an even number, a change of the n-th-order differential signal to a negative value and a positive value is repeated as many times as floor{(n+1)/2}, and then the n-th-order differential signal changes to a negative value. In other words, if the fact that a change in which the n-th-order differential signal becomes lower than the negative second threshold value and higher than the positive third threshold value is repeated as many times as floor{(n+1)/2} is detected, a change in contact condition can be determined even when the n-th-order differential signal is used. Note that floor (x) is the floor function of x.

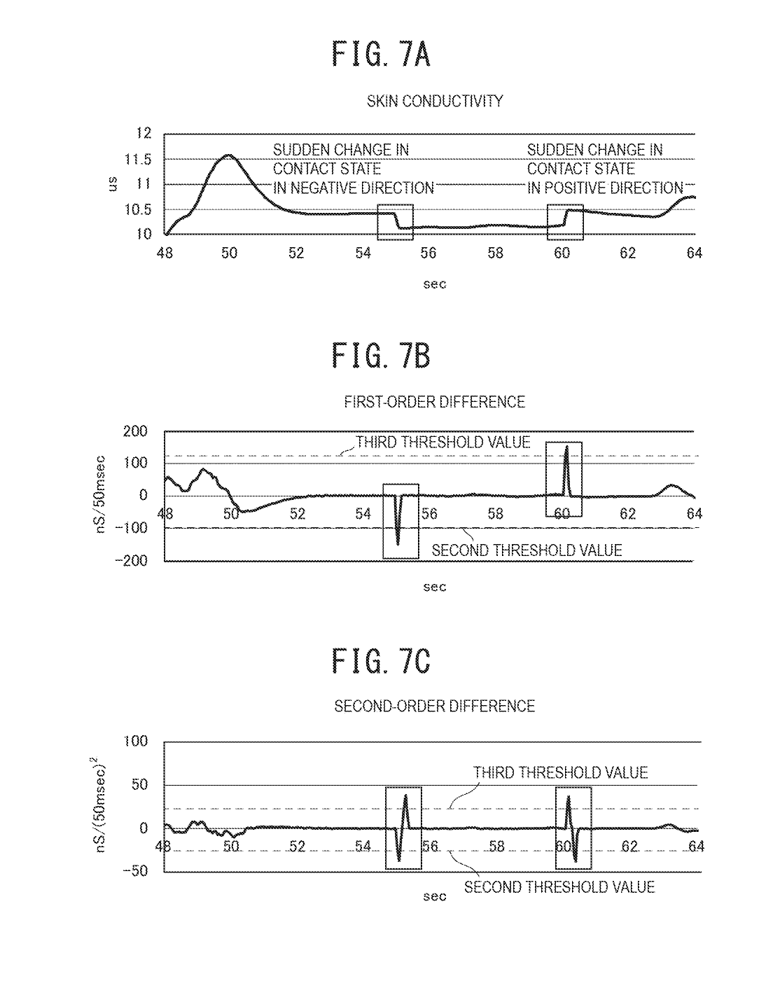

FIGS. 7A to 7C are waveform charts of outputs of the skin conductivity, the first-order differential signal, and the second-order differential signal when the contact condition between the skin and the electrode changes, where FIG. 7A is a chart illustrating the skin conductivity, FIG. 7B is a chart illustrating the first-order differential signal and the output of the emotional change estimating unit, and FIG. 7C is a chart illustrating the second-order differential signal and the output of the emotional change estimating unit. The charts illustrate the waveforms of the skin conductivity, the first-order differential signal, and the second-order differential signal when the state is changed from contacted state (a state where the electrodes and the skin are firmly stuck together) to substantially out of contact (a state where the electrodes and the skin are out of contact, or a state where the electrodes and the skin are weakly stuck together), and changed to the state of contact again after the state of being out of contact (being weakly stuck together) is continued for a while.

Specifically, the values are the measured values of skin conductivity, the first-order differential value, and the second-order differential value measured when the palm is turned up and down, or the like while wearing the skin conductivity measuring apparatus on the wrist.

The facing direction of the palm is changed with respect to the direction of gravity, and the skin conductivity measuring apparatus also is turned to the side of the Earth's surface of the wrist or to the opposite side. In such an occasion, the contact pressure between the skin and the electrode is changed due to the own weight of the skin conductivity measuring apparatus, and hence the base value of skin conductivity changes. In the case of such an artifact of change in contact condition, abrupt value changes in the order of positive direction and the negative direction are observed to be slightly separated in terms of time in the first-order difference of skin conductivity. In the second-order differential signal, two pairs of abrupt value changes in the positive and the negative direction are observed while the each order of value changes is different between the two pairs.

Even such a situation can be determined by using the first-order differential signal or the second-order differential signal of skin conductivity. When the n-th-order differential signal is used, a change in contact condition may be determined using two threshold values from both the change in the positive direction and the change in the negative direction, or from either of the changes. In this case, the first determination unit 21 may set either of the second threshold value and the third threshold value to 0 to determine the change in contact condition. Here, when the first determination unit 21 determines that the contact condition has changed, the emotional change estimating unit 15 may further correct data on the n-th-order differential signal at a corresponding first period to generate a second signal in order to estimate an emotional change based on the second signal. For example, the emotional change estimating unit 15 only has to correct the data on the n-th-order differential signal at the first period based on the n-th-order differential signal at a period other than the first period.

Skin conductivity data or differential value data in a portion determined to be a contact failure should not be used to determine the emotional change. Therefore, it is desired to correct the skin conductivity data or the differential value data in some way in order to estimate an emotional change using the corrected data. There are various correction methods. For example, a measured value during a period determined to be an artifact due to a contact failure may be replaced with a measured value determined to be valid during another period before the period, a measured value previously determined to be valid may not be updated until it is determined that the state of a contact failure is overcome when the contact failure is estimated, interpolation processing may be performed using a measured value during a period, previously determined to be an artifact due to a contact failure or a contact change, and a measured value determined to be valid during a period subsequent to the determined period, or the like. The above-mentioned methods or other various interpolation/correction methods can be applied depending on the responsiveness, the method for utilizing a measured value of skin conductivity, and the usage scene.

When instantaneousness is not required, data previous in terms of time may also be invalidated. In the case of a change from the state of being out of contact with the skin to the state of coming into contact with the skin, or when the contact between the electrode and the skin is being tighter due to a firm grip or the movement of the arm or the wrist, the skin conductivity changes in the positive direction (abnormal contact). Such a change is steeper than a change in skin conductivity associated with a human emotional change (phasic), and a differential value between adjacent measured values is larger than that of skin conductivity. Therefore, the change can be evaluated using threshold values larger in both the positive direction and the negative direction to distinguish from a change in measured value associated with a contact failure.

It is also useful to make an evaluation using the duration of an n-th-order differential value in order to determine a contact failure or an abnormal contact. The duration of a change in skin conductivity due to the contact failure or the abnormal contact is a short period. Since the duration is shorter than the time to change the skin conductivity due to an emotional reaction, a signal of the n-th-order differential value of skin conductivity results in changing from negative to positive, or from positive to negative in a short period of time. Therefore, as a result of evaluating the duration of a negative signal or a positive signal, if an event of a negative signal or a positive signal with duration shorter than a predetermined threshold value occurs, it can be determined to be a contact failure or an abnormal contact. When an n-th-order differential signal is evaluated to avoid the influence of noise, the amplitude may be determined appropriately with a threshold value so that a value equal to or larger than a predetermined amplitude threshold value will be adopted.

Although there are various methods of evaluating the duration of a signal, it is convenient to evaluate the time of a section from when the signal amplitude is zero until the next zero in either analog or digital form (zero cross evaluation).

### Embodiment 3

An emotional change estimating apparatus of Embodiment 3 is such that, in the emotional change estimating apparatus 20 of Embodiment 2 mentioned above, the first determination unit 21 determines that the contact condition between the skin and the electrode 1a, 1b has changed when a change in which the n-th-order differential signal b is higher than the positive fourth threshold value and lower than the negative fifth threshold value is repeated as many times as a value obtained by applying the floor function (n+1)/2.

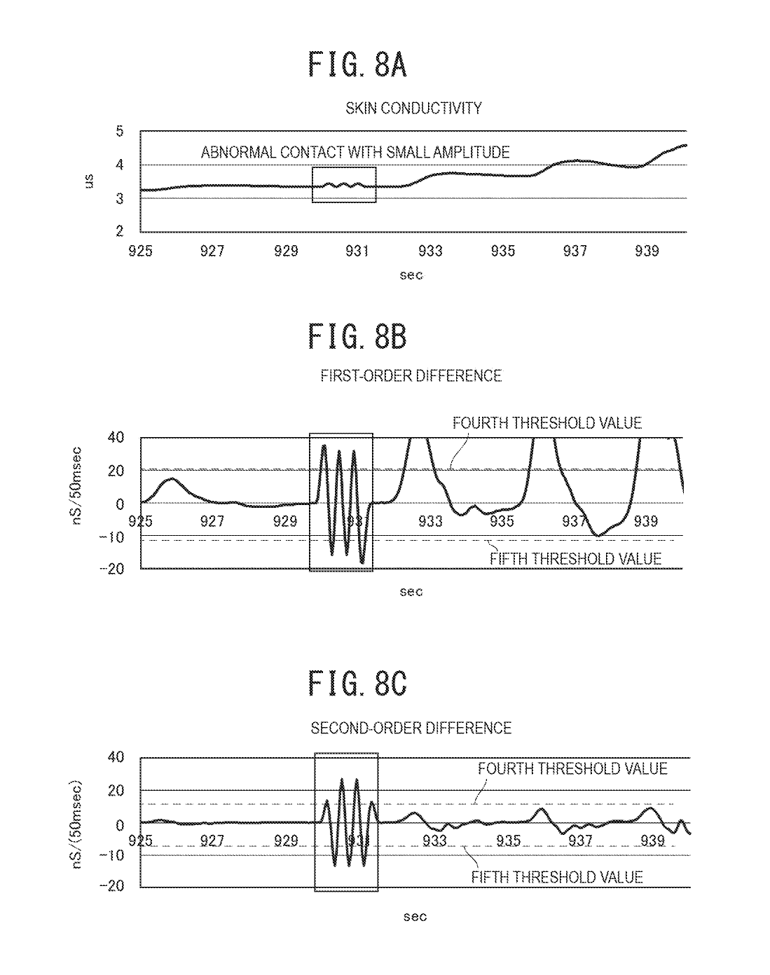

FIGS. 8A to 8C are waveform charts of outputs of the skin conductivity, the first-order differential signal, and the second-order differential signal when the contact condition between the skin and the electrode repeats a contact failure with a small amplitude, where FIG. 8A is a chart illustrating skin conductivity, FIG. 8B is a chart illustrating the output of the first-order differential signal, and FIG. 8C is a chart of the output of the second-order differential signal. In other words, these are waveform charts of the skin conductivity, the first-order differential signal, and the second-order differential signal when a contact failure is repeated with a small amplitude.

Here, when the first determination unit 21 determines that the contact condition has changed, the emotional change estimating unit 15 may further correct data on the n-th-order differential signal b at a corresponding first period to generate a second signal c in order to estimate an emotional change based on the second signal c. For example, it is only necessary for the emotional change estimating unit 15 to correct the data on the n-th-order differential signal b at the first period based on the n-th-order differential signal b at a period other than the first period.

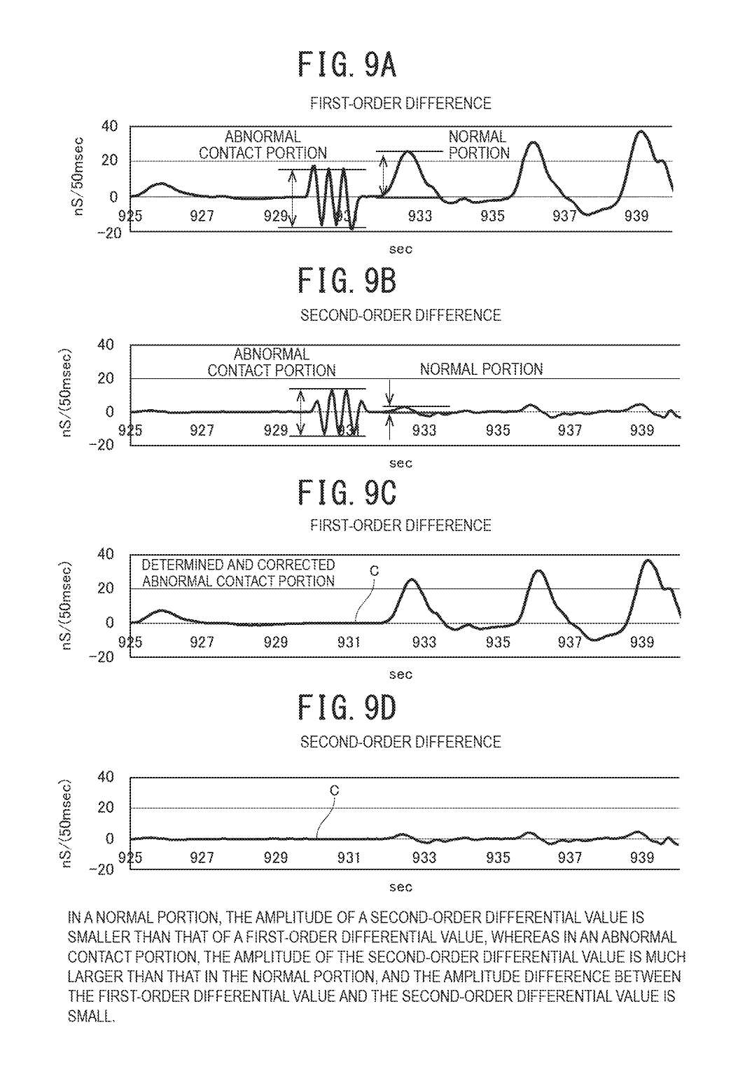

FIGS. 9A to 9D are waveform charts of outputs of the first-order differential signal and the second-order differential signal before and after the correction of a contact failure portion between the skin and the electrode, where FIG. 9A is a chart illustrating the first-order differential signal and the output of the emotional change estimating unit before the correction of the contact failure portion, FIG. 9B is a chart illustrating the second-order differential signal and the output of the emotional change estimating unit before the correction of the contact failure portion, FIG. 9C is a chart illustrating the first-order differential signal and the output of the emotional change estimating unit after the correction of the contact failure portion, and FIG. 9D is a chart illustrating the output of the second-order differential signal after the correction of the contact failure portion.

An artifact is generated not only due to a contact failure but also when the contact between the electrode and the skin is tightened by an action such as bending the wrist (abnormal contact condition). In this case, the artifact is observed as such an event that the measured value of skin conductivity first rises sharply and then drops sharply. The order of signal changes is opposite to that in the case of a contact failure. Even in this case, the first-order differential value or the second-order differential value of the measured value can be used to make an evaluation with a suitable threshold value in the same manner to determine an abnormal contact condition, and the above-mentioned correction can also be applied. Further, since the order of signal changes, i.e., a pair of a sharp rise and a sharp drop of the first-order differential value of skin conductivity are observed, the abnormal contact can be determined. In the case of the second-order differential value, the value further rises sharply after the sharp rise and the sharp drop. When signal changes occur in such order with large amplitudes, a contact failure can be determined.

Embodiment 3 can also correct data as in FIGS. 9C and 9D by the same means as in Embodiment 2 mentioned above.

### Embodiment 4

An emotional change estimating apparatus of Embodiment 4 is such that, in the emotional change estimating apparatus 10 of Embodiment 1 mentioned above, the emotional change estimating unit 15 changes the first threshold value according to a digital signal (first signal). Specifically, the emotional change estimating unit 15 changes the first threshold value so that the first threshold value shall be a monotonic function of an average value of the first signal.

Although information indicating that there is an SCR reaction is output to the outside, various methods are considered to output, to the outside, information as to whether it is significant as the SCR, such as to evaluate the first-order differential value with a predetermined threshold value and output a flag to the outside as an electric signal or information, where the flag indicates that there is an emotional change during a period of exceeding the threshold value, or output a pulsed flag at the point of exceeding the threshold value. A consideration to be noted in this case is the influence of the SCL value on the SCR change value.

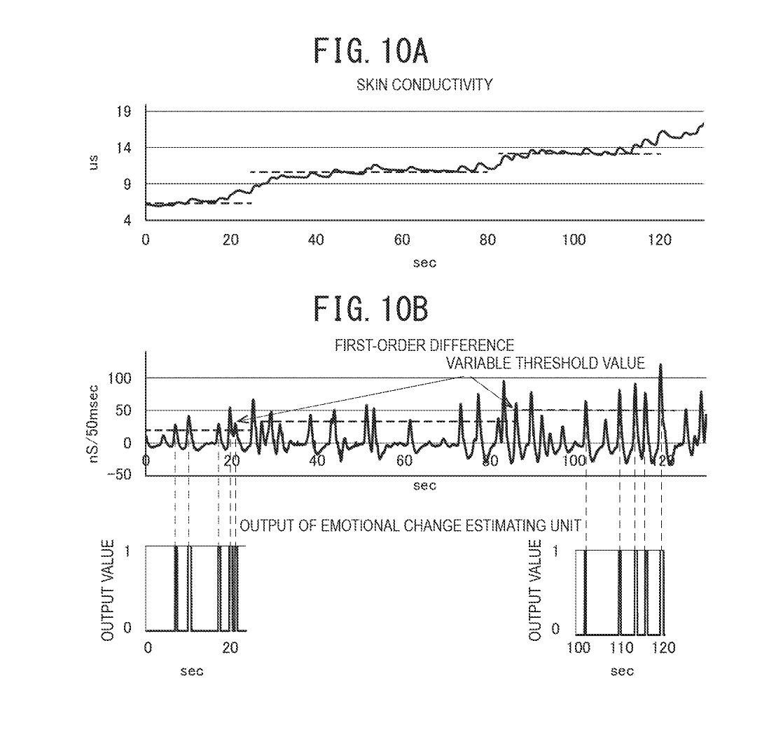

FIGS. 10A and 10B are waveform charts of the skin conductivity, the first-order differential signal, and the output of the emotional change estimating unit when the skin conductivity increases, where FIG. 10A is a chart illustrating the skin conductivity and FIG. 10B is a chart illustrating the first-order differential signal and the output of the emotional change estimating unit.

The emotional change estimating unit 15 in the emotional change estimating apparatus of Embodiment 4 is to change the first threshold value according to the first signal a. Further, the emotional change estimating unit 15 is to change the first threshold value so that the first threshold value shall be a monotonic function of an average value of the first signal a.

As will be apparent from FIG. 10B, the amount of SCR change tends to be observed to be small when the SCL level is low and to be large when the SCL is high. Therefore, as in FIG. 10B, a threshold value for an SCR change is preferably changed according to the SCL value.

Although the threshold value changes monotonously with respect to the SCL, it is changed as a nonlinear change. The reason is first that, when the SCL value is large, there are cases not only where a test subject is emotionally in an uplifted state, but also where the test subject sweats a lot due to the influence of environment temperature or humidity, and in the latter case, even if the SCL indicates a large value of 40 microsiemens or the like, the SCR often changes according to the SCL. Therefore when the SCL is high it is preferred to make an adjustment so that the rate of change in the threshold value for the SCL will get smaller than the tendency of an SCL-dependent change of the threshold value in case of the SCL is smaller.

When the direct-current level (Tonic) of skin conductivity is high, the amount of change (Phasic) in skin conductivity associated with the emotional change becomes large. Therefore, it is preferred to change the threshold value for evaluating a change in skin conductivity according to the direct-current level. The direct-current level may be obtained by taking a long-term average of measured values, or by using a representative value of measured values when such a phenomenon that the n-th-order difference of adjacent measured values is less than or equal to a predetermined threshold value appears continuously a fixed number of times. A measured value when the n-th-order difference becomes zero may also be selected as the direct-current level.

In Embodiment 4, not only is a differential value of skin conductivity determined to obtain an index of change in emotional state, but also the differential value can be used to estimate a state where there is no emotional change in order to obtain a direct-current level from the value at that time without a complicated circuit configuration.

Further, a measured value used to evaluate a change in electric conductivity may be used as the base without estimating the direct-current level. Since the amount of change in skin conductivity associated with an emotional change is often a small value compared with the direct-current level of the measured value, and then an error of the direct-current level is small enough even when a value associated with the change is superimposed on the direct-current level, the amount of change in skin conductivity does not give a large error to a threshold value used to evaluate the skin conductivity. The threshold value used to evaluate the skin conductivity may be changed appropriately using a value of either of adjacent measured values for evaluating a change in electric conductivity, or an average value of the measured values, or a numeric value derived by another method.

In Embodiment 4 mentioned above, the way of changing the first threshold value according to the first signal a is described. This is exactly equivalent to a case where a value of the n-th-order differential value is normalized using the direct-current level without changing the threshold value.

### Embodiment 5

FIG. 11 is a block diagram for describing Embodiment 5 of an emotional change estimating apparatus according to the present invention. In the figure, reference symbol 14b denotes an m-th-order differential signal output unit, 22 is a second determination unit, and 30 is an emotional change estimating apparatus. Note that constituent features having the same functions as those in FIG. 2 are given the same reference symbol s.

The emotional change estimating apparatus 30 of Embodiment 5 is an emotional change estimating apparatus for measuring the electrodermal activity of a living body to estimate a change in excited state of this living body, including an electrophysical quantity measuring unit 12, a differential signal output unit 14, an emotional change estimating unit 15, and a second determination unit 22.

The differential signal output unit 14 is composed of an n-th-order differential signal output unit 14a and an m-th-order differential signal output unit 14b.

The m-th-order differential signal output unit 14b outputs, to a data accumulation unit 13, an m-th-order differential signal d, (where m is an integer larger than n) of the first signal a, and the m-th-order differential signal d is input from the data accumulation unit 13 to the second determination unit 22. In this example, the n-th-order differential signal b and the m-th-order differential signal d are input to the second determination unit through a data bus from the data accumulation unit 13 to the second determination unit 22.

Based on the n-th-order differential signal b and the m-th-order differential signal d, the second determination unit 22 determines whether the first signal a contains a noise signal caused by a change in contact condition between the electrode 1a, 1b and the skin.

Further, the second determination unit 22 determines that the noise signal is contained when the ratio between the amplitude of the m-th-order differential signal d and the amplitude of the n-th-order differential signal b is larger than a sixth threshold value, and determines that no noise signal is contained when the ratio between the amplitude of the m-th-order differential signal d and the amplitude of the n-th-order differential signal b falls in a range of values smaller than a seventh threshold value.

When the second determination unit 22 determines that the noise signal is contained, the emotional change estimating unit 15 corrects data on the n-th-order differential signal b at a corresponding second period to generate a third signal e in order to estimate an emotional change based on this third signal e.

Further, based on the n-th-order differential signal b at a period other than the second period, the emotional change estimating unit 15 corrects data on the n-th-order differential signal b at the second period, where the integer m of the m-th-order differential signal d is m=2.

In other words, the emotional change estimating apparatus 30 of Embodiment 5 includes the second determination unit 22 in the emotional change estimating apparatus 10 of Embodiment 1 mentioned above, where the differential signal output unit 14 further outputs the m-th-order differential signal (where m is an integer larger than n) of a digital signal. The data accumulation unit 13 holds the n-th-order differential signal b and the m-th-order differential signal d, and outputs these two signals to the second determination unit 22.

Here, in the emotional change estimating apparatus 30 of Embodiment 5, waveform charts of the outputs of the skin conductivity, the first-order differential signal (the n-th-order differential signal b in the case of n=1), and the second-order differential signal (the m-th-order differential signal in the case of m=2) when a contact failure in the contact condition between the skin and the electrode is repeated with a small amplitude are the same as those of FIGS. 8A to 8C.

When a change in skin conductivity associated with an emotional change is evaluated with a second-order differential value, it takes a value in the positive direction, then a value in the negative direction, and further a value in the positive direction, but the amplitude is small as a whole. This is because the emotional change that causes the change in skin conductivity is a slow change. On the other hand, a change in skin conductivity caused by a contact failure is a steep change compared with that associated with the emotional change, and the duration of the changing state is short. Therefore, when the change in skin conductivity caused by the contact failure is evaluated with the second-order differential value, the amplitude is observed to be larger than that associated with the emotional change. From another perspective, the change in skin conductivity associated with the emotional change is so observed that the amplitude of the second-order differential value is smaller than the amplitude of the first-order differential value, whereas the change in skin conductivity caused by the contact failure is so observed that a difference between the amplitude of the first-order differential value and the amplitude of the second-order differential value is small. In some cases, the amplitude of the second-order differential value may be larger than the amplitude of the first-order differential value.

The same is true in the case of a change from the state of being out of contact with the skin to the state of coming into contact with the skin and in the case where the contact between the electrode and the skin is made tighter due to a firm grip or the movement of the arm or the wrist.

Further, in such a situation that a contact failure, which is triggered by motion (shaking) such as to rotate/shake the hand quickly, and a re-contact is repeatedly continued, the amplitude of the first-order differential value of the change in skin conductivity is observed to be relatively small, and it may be difficult to distinguish from the emotional change. However, if the second-order differential value of the change in skin conductivity is evaluated, it will be an amplitude of the same level as or larger than the amplitude of the first-order difference, and hence it is easy to distinguish from the change in skin conductivity caused by the emotional change.

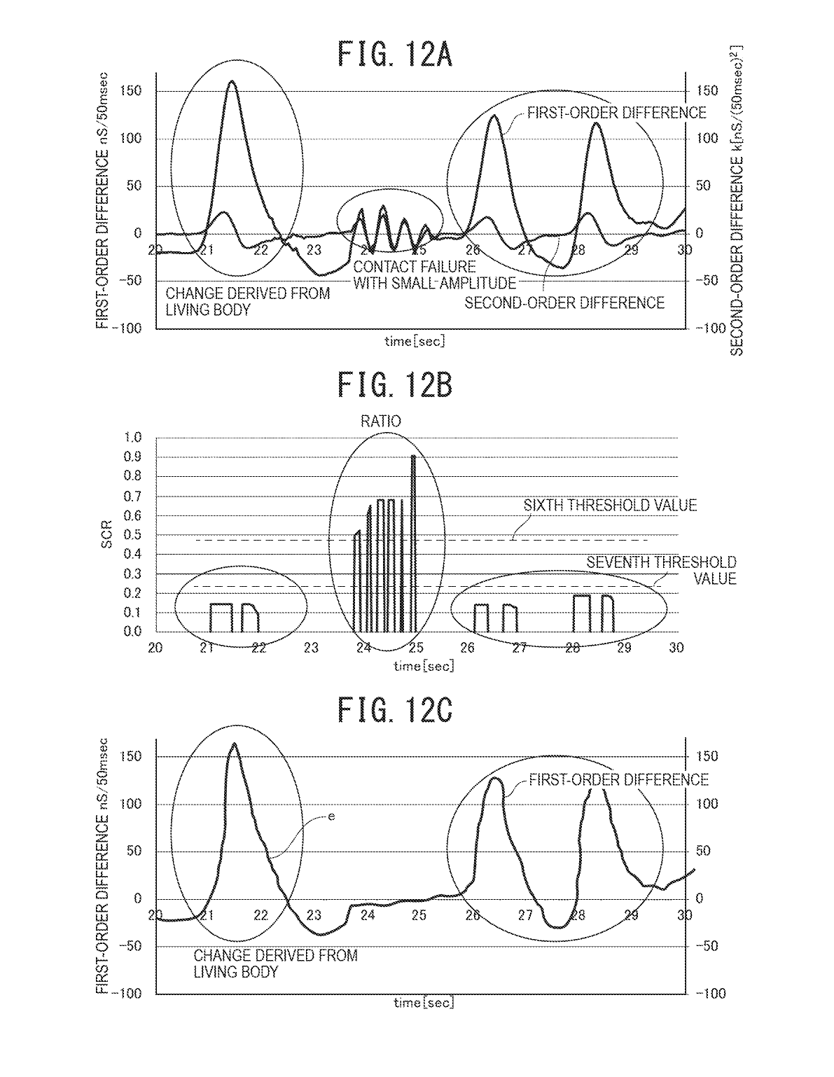

FIGS. 12A to 12C are charts for comparing amplitude differences between the first-order difference and the second-order difference with and without a contact failure. In terms of an artifact due to a change in this contact condition, it is found that the amplitude difference between the first-order differential value and the second-order differential value is smaller than the signal change associated with the emotional change. Even in the artifact superimposed on the measured value of skin conductivity measured when the palm is turned up and down as illustrated in FIGS. 7A to 7C, it is also found that the amplitude difference between the first-order differential value and the second-order differential value in the artifact portion is smaller than the amplitude difference between the first-order differential value and the second-order differential value in a signal change associated with an emotional change. It is further found that the similar is observed in the sudden contact failure which shows continuous change of positive and negative direction as illustrated in FIG. 6A.

In other words, it is useful to evaluate amplitudes of the first-order differential value and the second-order differential value of the measured value of skin conductivity and determine a contact failure or a change in contact condition such as an abnormal contact based on a difference between the amplitudes or a ratio of the amplitudes.

Further, when the second determination unit determines that a noise signal is contained, i.e., when a signal indicating that a noise signal is contained is input from the second determination unit, the emotional change estimating unit 15 further corrects data on the n-th-order differential signal b at a corresponding second period to generate a third signal e illustrated in FIG. 12C in order to estimate an emotional change based on this third signal e.

As illustrated in FIG. 12A, when an evaluation is made with the first-order differential value of the change in skin conductivity associated with the emotional change, changes in the negative direction and then in the positive direction, or changes in reverse order are indicated. In addition, the amplitude values of the change in the negative direction and the change in the positive direction are nearly equal. This reflects that the change in skin conductivity caused by a contact failure often results from body motion, walking, or the like, and the rate of such a motion change is a constant value. On the other hand, the emotional change that causes a change in skin conductivity falls more slowly than the rising, and the first-order differential values lack symmetry. Therefore, the symmetry of a signal in the adjacent positive direction and negative direction is so evaluated that a contact failure or an abnormal contact can be determined from a vital signal. The above description is not limited to the first-order differential value, and the same is true in the case of evaluating the n-th-order differential value.

Further, based on the n-th-order differential signal b at a period other than the second period, the emotional change estimating unit 15 corrects data on the n-th-order differential signal b at the second period.

Skin conductivity data or differential value data on a portion determined to be a contact failure cannot be used to determine the emotional change. Therefore, it is desired to correct the skin conductivity data or the differential value data in some way in order to estimate an emotional change using the corrected data. There are various correction methods. For example, a measured value during a period determined to be an artifact due to a contact failure may be replaced with a measured value determined to be valid during another period before the period, a measured value previously determined to be valid may not be updated until it is determined that the state of a contact failure is overcome when the contact failure is estimated, interpolation processing may be performed using a measured value during a period, previously determined to be an artifact due to a contact failure or a contact change, and a measured value determined to be valid during a period subsequent to the determined period, or the like. The above-mentioned methods or other various interpolation/correction methods can be applied depending on the responsiveness, the method for utilizing a measured value of skin conductivity, and the usage scene.

When instantaneousness is not required, data previous in terms of time may also be invalidated. In the case of a change from the state of being out of contact with the skin to the state of coming into contact, or when the contact between the electrode and the skin is made tighter due to a firm grip or the movement of the arm or the wrist, the skin conductivity changes in the positive direction. Such a change is steeper than a change (phasic) in skin conductivity associated with a human emotional change, and a differential value between adjacent measured values is larger than that of skin conductivity. Therefore, the change can be evaluated using threshold values larger in both the positive direction and the negative direction to distinguish from a change in measured value associated with a contact failure. Even in Embodiment 5, data can be corrected as in FIGS. 8C and 8D.

Note that FIGS. 12A and 12B illustrate an example when peak values p1 and p2 of the first-order difference and second-order difference having a predetermined width in back and forth of certain time t are extracted respectively to calculate the ratio of them.

In Embodiment 5 mentioned above, a change in skin conductivity associated with an emotional change in the negative direction is slower than a change in the positive direction. Therefore, threshold values for evaluating a differential value may be different between the positive one and the negative one. When the contact condition becomes instantaneously failure as illustrated in FIGS. 6A to 6C, both the change in the positive direction and the change in the negative direction are steep. The measurement time interval needs to be a time interval capable of capturing a steep change in the positive direction. The change in the positive direction lasts for about one second or more, and the following change in the negative direction lasts for about a few seconds. However, in the case of a continuous stimulus, a change in the following positive direction may occur in the middle of the change in the negative direction. In general, there is a time lag of about 0.5 seconds or more from the stimulus to the response of skin conductivity. Therefore, a change of at least 0.5 seconds or less should be detected in order to capture the change in skin conductivity in real time.

Preferably a measurement shall be done at least every 200 ms (five samples per second), or every 100 ms (five samples per 0.5 seconds). The measurement may also be done more frequently. And using a calculated value such as the average value, an evaluation can be performed at every 100 ms, for example.

In other words, it is not necessarily to use adjacent values in raw data on the measured values to evaluate a differential value. An effective measured value can be calculated from measured values and the differential value can be derived from adjacent effective values. For example, in the case of making a measurement every 50 ms, it is feasible to evaluate, as effective data, every two or four measured values, rather than a difference between consecutive measured values. Further, it is considered a method of taking a moving average or block average of every two or four measured data to have an evaluation. This moderates the requirement of the resolution and noise in the measurement system. Under the conditions of high temperature and humidity, the SCR reaction occurs more frequently, and when the SCL is large, the SCR tends to be larger. In such a case, measured values may be evaluated every 50 ms. However, in the case of low humidity and low temperature, or in the case of a test subject with a small SCL, every two or four measured values can be evaluated. Needless to say, the same effect can be obtained by using a method of changing the measurement interval according to the SCL value or the environment. Note that these methods can be applied to Embodiment 1 mentioned above in the same manner.

It is desired that any artifact caused by a change in contact condition should be removed and corrected from the effective measured value mentioned above.

### Embodiment 6

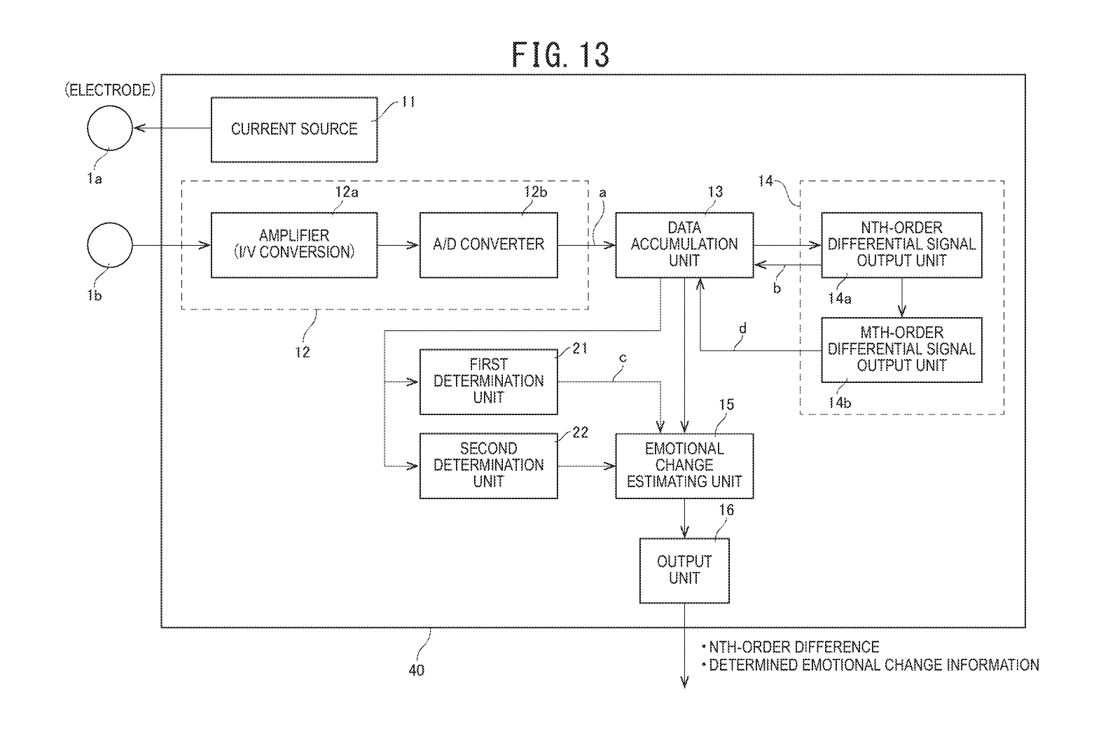

FIG. 13 is a block diagram for describing Embodiment 6 of an emotional change estimating apparatus according to the present invention. Constituent features having the same functions as those in FIG. 5 and FIG. 11 are given the same reference symbols. In each of the aforementioned embodiments, the differential signal output unit for outputting the m-th-order differential signal, and the first and second determination units may be provided.

An emotional change estimating apparatus 40 of Embodiment 6 is an emotional change estimating apparatus for measuring the electrodermal activity of a living body to estimate a change in excited state of this living body, including an electrophysical quantity measuring unit 12, a differential signal output unit 14, an emotional change estimating unit 15, a first determination unit 21, and a second determination unit 22.

Based on the n-th-order differential signal b, the first determination unit 21 determines whether the contact condition between the skin and the electrode 1a, 1b has changed. Further, based on the n-th-order differential signal b and the m-th-order differential signal d, the second determination unit 22 determines whether the first signal a contains a noise signal caused by a change in contact condition between the electrode 1a, 1b and the skin.

### Embodiment 7

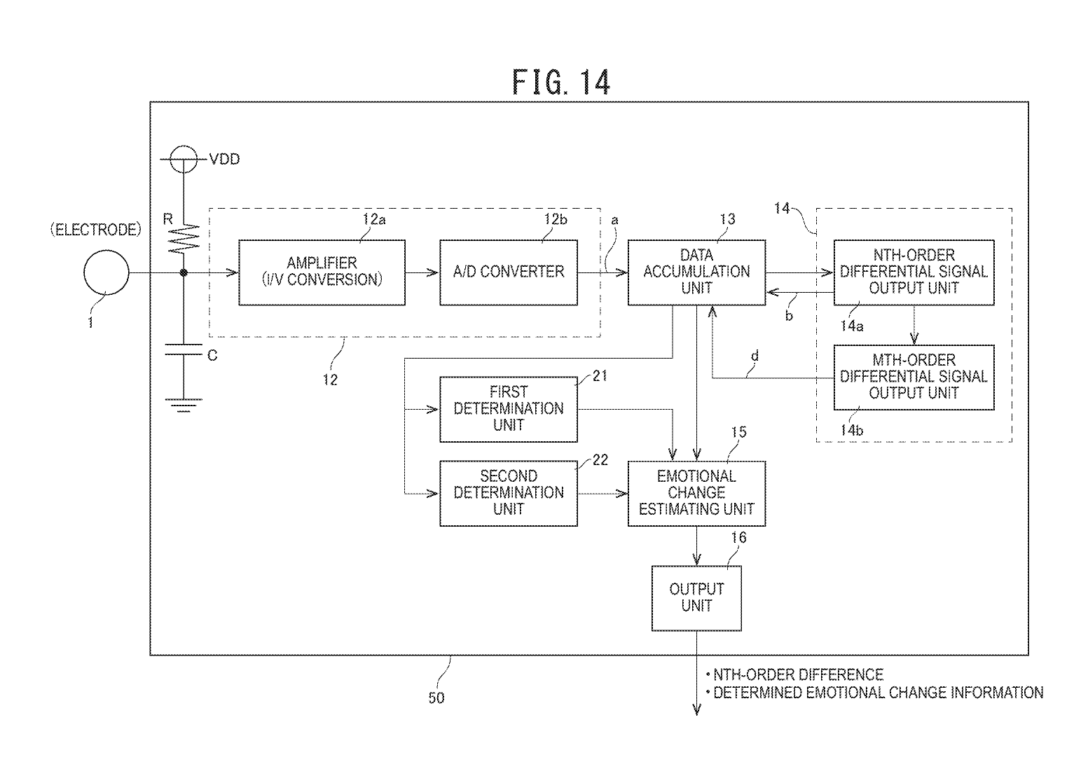

FIG. 14 is a block diagram for describing Embodiment 7 of an emotional change estimating apparatus according to the present invention. Constituent features having the same functions as those in FIG. 13 are given the same reference symbols.

An emotional change estimating apparatus 50 of Embodiment 7 is an emotional change estimating apparatus for measuring the electrodermal activity of a living body to estimate a change in excited state of this living body in order to detect an emotional change by a capacitance measurement through a contact with the skin of the living body.

Even in this case, it is required to detect a change in capacitance due to sweat or the like associated with an emotional change regardless of a change in baseline, and determine an artifact due to a contact changing state such as a contact failure or an abnormal contact in order to correct the data. Therefore, the present invention mentioned above can be applied to make a capacitance measurement to detect an emotional change.

Of course, this apparatus can also be widely applied to measurements of electrophysical quantities related to vital signals such as the skin resistance, the skin potential, the pulse wave, the brain wave, and EEG.

Although the embodiments of the present invention have been described with respect to measured values of skin conductivity, it is needless to say that the present invention can also be applied to a case where any electrophysical quantity signal of a living body is measured other than the skin conductivity.

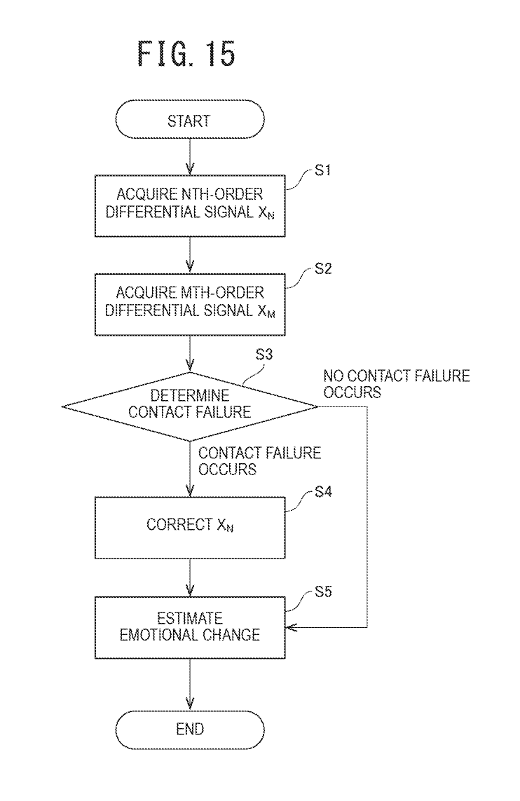

FIG. 15 is a flowchart for describing an emotional change estimating method according to the present invention. The emotional change estimating method of the present invention is an emotional change estimating method for measuring the electrodermal activity of a living body to estimate a change in excited state of this living body.

First, the method includes measuring an electrophysical quantity on at least one of electrodes 1a and 1b adapted to come into contact with the skin of the living body and outputting a first signal a corresponding to this electrophysical quantity (electrophysical quantity measuring step).

Next, the method includes inputting data on the first signal a at multiple times, acquiring an n-th-order differential signal (b=Xn, where n is an integer of 1 or more) of the first signal a, and acquiring an m-th-order differential signal (d=Xm, where m is an integer larger than n) of the first signal a (differential signal output step (S1, S2)). This is a step of outputting an n-th-order time differentiation signal of the first signal a.

Next, the method includes determining whether the contact condition between the skin and the electrode 1a, 1b has changed based on the n-th-order differential signal Xn, and determining whether the first signal a contains a noise signal caused by a change in contact condition between the electrodes 1a, 1b and the skin (determination step (S3)) based on the n-th-order differential signal Xn and the m-th-order differential signal Xm.

Next, the method includes correcting the n-th-order differential signal (Xn) when a contact failure occurs in the determination step S3 (correction step (S4)).

Next, the method includes estimating an emotional change of the living body based on output from the correction step S4 (emotional change estimating step (S5)).

If the intended purpose is to output data after a noise signal associated with a change in contact condition is removed, the method may be ended at the correction step S4.

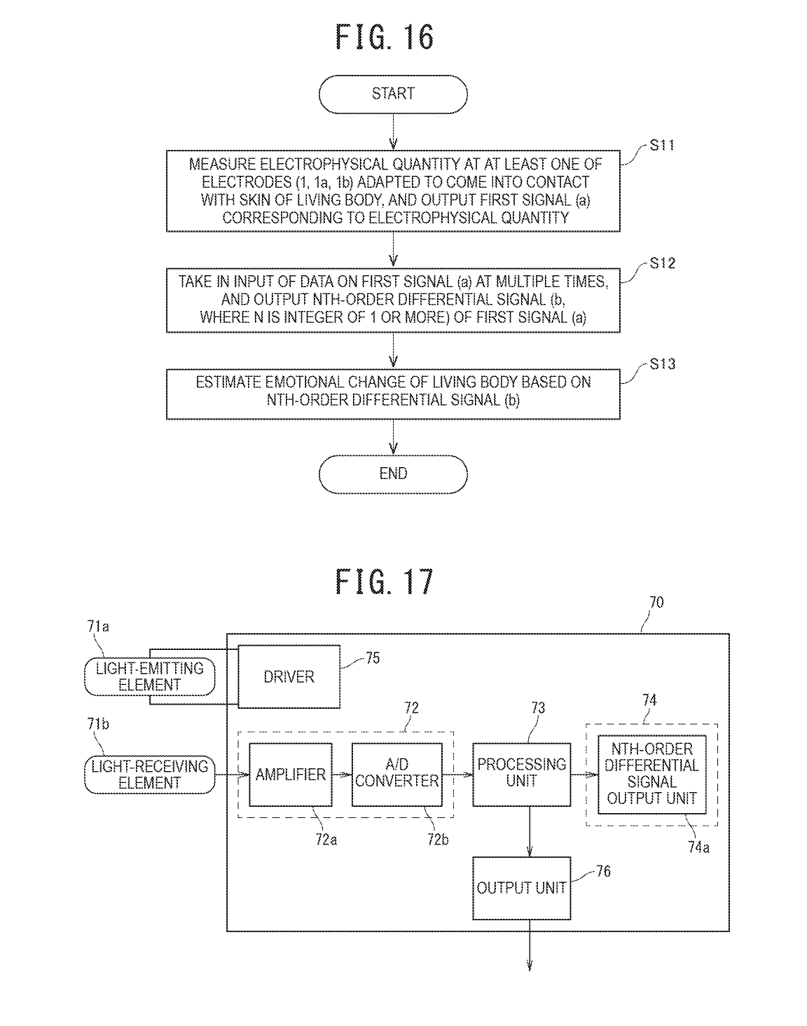

FIG. 16 is another flowchart for describing another emotional change estimating method according to the present invention. The other emotional change estimating method of the present invention is an emotional change estimating method for measuring the electrodermal activity of a living body to estimate a change in excited state of the living body, which includes: first measuring an electrophysical quantity on at least one of electrodes 1a and 1b adapted to come into contact with the skin of the living body, and outputting a first signal a corresponding to this electrophysical quantity (electrophysical quantity measuring step (S11)); inputting next data on the first signal a at multiple times, and outputting an n-th-order differential signal b, where n is an integer of 1 or more) of the first signal a (differential signal output step (S12)); and next estimating an emotional change of the living body based on the n-th-order differential signal b from the differential signal output step (emotional change estimating step (S13)). The electrophysical quantity measuring step S11 is a step of measuring a vital signal and outputting a first signal a corresponding to the vital signal. The differential signal output step S12 is a step of outputting an n-th-order time differentiation signal (where n is an integer of 1 or more) of the first signal a.

The emotional change estimating step is to compare the n-th-order differential signal b with a first threshold value in order to estimate an emotional change of the living body.

Further, the method includes determining whether the contact condition between the skin and the electrode 1a, 1b has changed (first determination step) based on the n-th-order differential signal b. The first determination step is a step of determining the contact condition between a vital signal measuring apparatus and the living body based on an n-th-order time differentiation signal.

Since the electrophysical quantity measuring step S11, the differential signal output step S12, and the first determination step are included, a contact condition estimating method for determining a contact condition of a vital signal measuring apparatus which measures a vital signal in contact with a living body can be realized.

This first determination step is to determine that the contact condition between the skin and the electrode 1a, 1b has changed when a change in which the n-th-order differential signal b is lower than a negative second threshold value and higher than a positive third threshold value is repeated as many times as a value obtained by applying a floor function (n+1)/2.

Further, the first determination step is to determine that the contact condition between the skin and the electrode 1a, 1b has changed when a change in which the n-th-order differential signal b is higher than a positive fourth threshold value and lower than a negative fifth threshold value is repeated as many times as the value obtained by applying the floor function (n+1)/2.

When the first determination step determines that the contact condition has changed, the emotional change estimating step is to correct data on the n-th-order differential signal b at a corresponding first period, generates a second signal c, and estimate an emotional change based on this second signal c.

Further, the emotional change estimating step is to correct the data on the n-th-order differential signal b at the first period based on the n-th-order differential signal b at a period other than the first period. Further, the emotional change estimating step is to change the first threshold value according to the first signal a. Further, the emotional change estimating step is to change the first threshold value so that the first threshold value shall be a monotonic function of an average value of the first signal a.

The differential signal output step further includes outputting an m-th-order differential signal d, (where m is an integer larger than n) of the first signal a, and determining whether the first signal a contains a noise signal caused by a change in contact condition between the electrode 1a, 1b and the skin (second determination step) based on the n-th-order differential signal b and the m-th-order differential signal d.

This second determination step is to determine that a noise signal is contained when the ratio of the amplitude of the m-th-order differential signal d and the amplitude of the n-th-order differential signal b is larger than a sixth threshold value, and determine that no noise signal is contained when the ratio of the amplitude of the m-th-order differential signal d and the amplitude of the n-th-order differential signal b is smaller than a seventh threshold value.

When it is determined in the second determination step that a noise signal is contained, the emotional change estimating step is to correct data on the n-th-order differential signal b at a corresponding second period, generate a third signal e, and estimate an emotional change based on this third signal e.

Further, the emotional change estimating step is to correct the data on the n-th-order differential signal b at the second period based on the n-th-order differential signal b at a period other than the second period.

Further, the first determination step of determining whether the contact condition between the skin and the electrode 1a, 1b has changed based on the n-th-order differential signal b, and the second determination step of determining whether the first signal a contains a noise signal caused by a change in contact condition between the electrode 1a, 1b and the skin are included based on the n-th-order differential signal b and the m-th-order differential signal d.

There is also provided a program for causing a computer to execute each of the above-mentioned steps based on an emotional change estimating method for measuring the electrodermal activity of a living body, and estimating a change in excited state of this living body. Further, there is provided a computer-readable recording medium on which the program for executing each of the above-mentioned steps is recorded.

The determination of a motion artifact by the method described in Embodiments 2, 3, and 5 can be applied to a case where biological information is measured by attaching a detector to a living body and using the measurement of an electrophysical quantity other than the skin conductivity, an optical method, or any other method. Of course, the same method mentioned above can also be applied to a skin resistance value as the reciprocal of skin conductivity. In addition, for example, it can be applied to a case where capacitance between the skin and electrodes is measured.

The method of measuring capacitance between the skin and the electrode is used to measure the skin moisture content or to determine the contact itself. In this application, the contact is stabilized at the DC value of a measured value. Even in this case, it is useful to use the n-th-order differential value of a measured value to determine whether the contact is stable or the contact condition is fluctuated. In addition, the embodiment can be applied to the skin potential, the myoelectric potential, the cardiac potential, or the like to measure, with electrodes, an electric signal or an electric state attributed to the living body, or the determination and processing of an artifact caused by a change in contact condition between detection elements and the skin in measuring the plethysmogram by an optical method.

In general, since a change in vital signal is slower than data sampling of an A/D converter, the n-th-order differential value of a time-series signal represents a small value in a vital signal but a large value in an artifact signal caused by a change in contact condition. Therefore, a threshold value for determining a change in contact condition with the n-th-order differential value can be set to be a larger value than a threshold value for determining a vital signal. Further, a threshold value for determining a contact failure and a threshold value for determining an abnormal contact do not have to be identical. In the case of occurrence of a contact failure, the contact failure may be caused by vibration while a test subject wearing a biological sensor is walking or taking exercise, whereas the abnormal contact may be caused by the movement of a living body such as to clench a fist. In this case, there may be a difference in the rate of signal change because of different events that cause the signal changes. In such a case a noticeable difference in signal amplitude appears in the n-th-order differential value of the vital signal, and then the threshold value can be changed between the contact failure and the abnormal contact, respectively. Further, since it is obvious that the threshold value varies according to the type of vital signal used, the characteristics and structure of the sensor, and the configuration of equipment including the sensor and worn on the living body, it would be better to set a threshold value accordingly.

FIG. 17 is a block diagram of a plethysmogram measuring apparatus in which light obtained by transmitting or reflecting light from a light-emitting element through or on a living body is received by a light-receiving element to obtain a pulsation signal from output of the light-receiving element. In the figure, reference symbol 70 denotes a plethysmogram measuring apparatus, 71a is a light-emitting element, 71b is a light-receiving element, 75 is a driver, 72 is a plethysmogram measuring unit, 72a is an amplifier, 72b is an A/D converter, 73 is a processing unit, 74 is a time differentiation signal output unit, 74a is an n-th-order differential signal output unit, and 75 is an output unit.

Since the intensity of transmitted light or reflected light varies with the light absorbing characteristics of blood in synchronization with the heartbeat, the heartbeat and the pulse wave can be detected from the intensity of the light.

The plethysmogram measuring apparatus 70 includes the driver 75 of the light-emitting element 71a, the amplifier 72a, the A/D converter 72b, the processing unit 73, and the output unit 76. The time differentiation signal output unit 74 takes in input of a first signal, including the n-th-order differential signal output unit 74a for outputting an n-th-order time differentiation signal (where n is an integer of 1 or more) of the first signal.

A blue, green, yellow, red, infrared, or white light-emitting diode, a semiconductor laser, a xenon lamp, or the like may be used for the light-emitting element 71a, and a photodiode or a phototransistor may be used for the light-receiving element 71b, or other practical elements can be used.

A pulsation signal converted to a time-series signal at the A/D converter 72b is subjected to filter processing and the like in the processing unit 73, and output from the output unit 76. Simultaneously, a differential signal is calculated in the n-th-order differential signal output unit 74a and output to the processing unit 73. Based on the n-th-order differential signal, the processing unit 73 determines whether the state is a contact failure state, and in the case of the contact failure state, the time-series signal is processed and then output.

Processing details include invalidating a signal in the contact failure state, replacing a portion in the contact failure state with past data at the same time interval, or when the pulsebeat is calculated in the processing unit 73, computing the pulse rate in consideration of a contact failure portion, and not acquiring data when the contact failure or the abnormal contact occurs in succession. In addition, other various processing tasks are performed to reduce the influence of the contact failure.

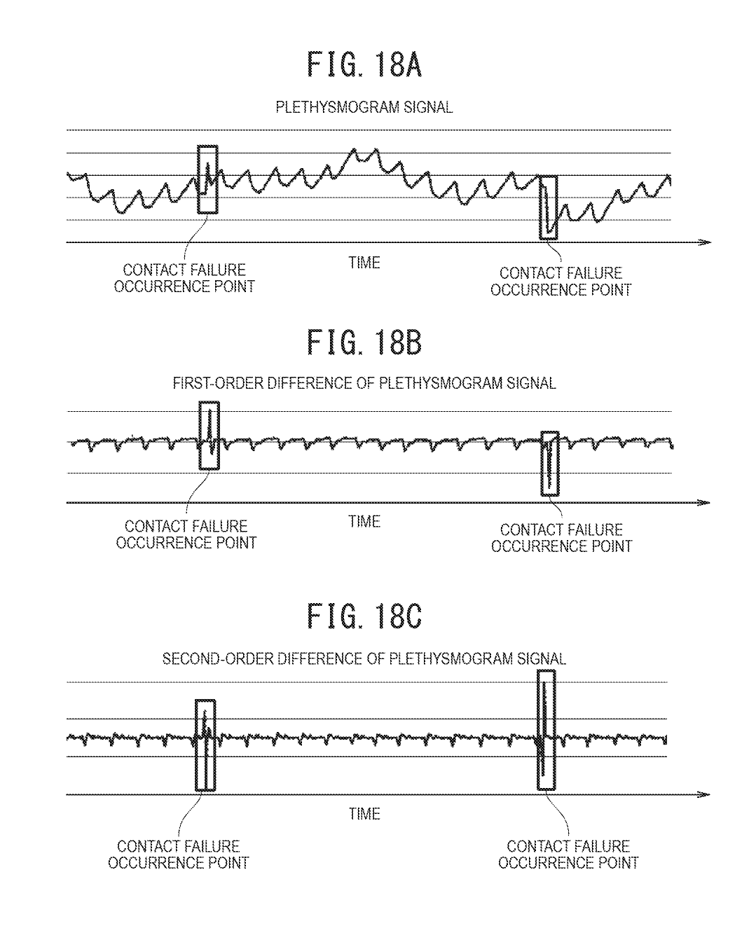

FIGS. 18A to 18C are waveform charts of an example of a plethysmogram signal including a measured artifact signal caused by a contact failure, illustrating the first-order differential signal and the second-order differential signal together. In the first-order differential signal and the second-order differential signal, the amplitude of the artifact signal becomes large, and it is found that the artifact signal can be determined by setting an appropriate threshold value.

As mentioned above, the present invention can be applied to various vital signals. FIG. 1 is a block diagram when the present invention is expanded to general vital signals, and the biological sensor in the figure may also be applied to various other biological sensors for electrocardiogram, electromyogram, body fat, and bioelectrical impedance, in addition to the sensors for skin conductivity and pulsebeat.

Further, in the case of equipment including multiple biological sensors, a signal from any one of the sensors can be used to determine the contact property by the above-mentioned method, or the contact property can also be determined using multiple sensor signals.

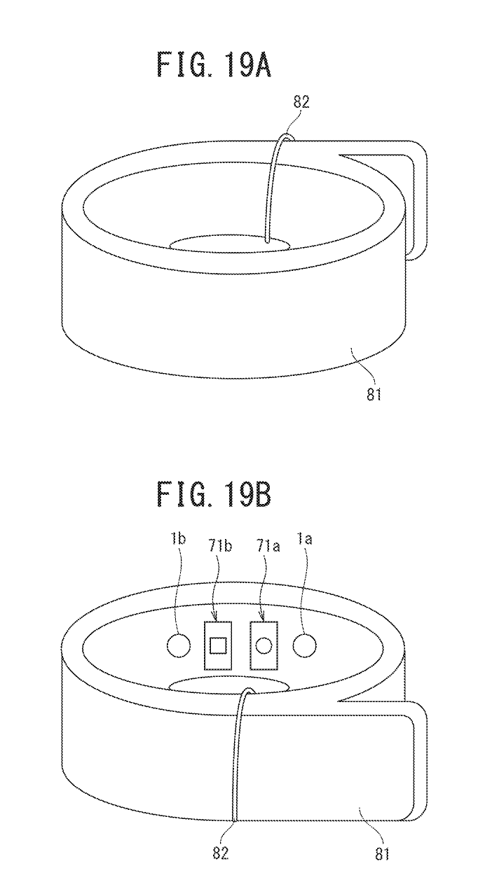

FIGS. 19A and 19B are diagrams illustrating a specific example of a sensor unit for realizing each of the aforementioned embodiments. In the figure, reference symbol 81 denotes an arm band and 82 is a fixing tool. Note that constituent features having the same functions as those in FIG. 2 and FIG. 17 are given the same reference symbols.

The sensor unit has a wristwatch shape capable of being worn on a user's arm or wrist, including skin conductivity sensors 1a, 1b, and optical plethysmogram sensors 71a, 71b in the main body.

Using the above-mentioned method, the skin conductivity sensors detect a change in skin conductivity, the pulse wave sensors detect a change in bloodstream due to the heartbeat, and information on a contact condition can be obtained from the n-th-order differential value of each detected signal. The contact condition may be determined using information obtained from the skin conductivity sensors or information obtained from the pulse wave sensors, or using both. In the state of a contact failure or an abnormal contact, acquired data are processed to get rid of or invalidate abnormal data, or not to energize a light source in order to enable applications such as to reduce current consumption.

As described above, the emotional change estimating apparatus and the emotional change estimating method according to the embodiments of the present invention particularly relate to technology for measuring a change in human skin conductivity to estimate an emotional change, and information on the estimated emotional change can be used in psychological applications and medical applications, and further in applications such as to control healthcare in the consumer field and to detect the reaction of a test subject to a game content.

In Patent Literature 1 mentioned above, since the least square method as statistical means is used and hence a change in skin conductivity of a living body cannot be detected at high speed, an emotional change of the living body cannot be estimated at high speed. In Patent Literature 2, the change in skin conductivity of the living body cannot be detected at high speed and hence the emotional change of the living body cannot be estimated at high speed as well as Patent Literature 1.

In the meantime, the emotional change estimating apparatus and the emotional change estimating method as one aspect of the vital signal measuring apparatus and the method for estimating a contact condition of the embodiment can detect a change in electrophysical quantity, such as skin conductivity, from the n-th-order differential signal at high speed, so that an emotional change of a living body can be estimated at high speed.

While the present invention has been described with reference to the specific embodiments, the description of the embodiments is not intended to limit the invention. It is obvious that those skilled in the art will appreciate other embodiments of the present invention together with various variations of the disclosed embodiments with reference to the description of the present invention. Therefore, the claims shall be interpreted to cover these variations or embodiments contained in the technical scope and gist of the present invention.

Embodiments of the above-mentioned emotional change estimating apparatus and emotional change estimating method will be described below.

### Embodiment 1

An emotional change estimating apparatus including: an electrophysical quantity measuring unit for measuring an electrophysical quantity on at least one electrode adapted to come into contact with the skin of a living body, and outputting a first signal corresponding to the electrophysical quantity;

a differential signal output unit for receiving data on the first signal at multiple times, and outputting an n-th-order differential signal (where n is an integer of 1 or more) of the first signal; and

an emotional change estimating unit for estimating an emotional change of the living body based on the n-th-order differential signal from the differential signal output unit.

### Embodiment 2

The emotional change estimating apparatus according to Embodiment 1, wherein the emotional change estimating unit compares the n-th-order differential signal with a first threshold value to estimate the emotional change of the living body.

### Embodiment 3

The emotional change estimating apparatus according to Embodiment 1 or 2, further including a first determination unit for determining whether a contact condition between the skin and the electrode has changed based on the n-th-order differential signal.

### Embodiment 4

The emotional change estimating apparatus according to Embodiment 3, wherein the first determination unit determines that the contact condition between the skin and the electrode has changed when a change in which the n-th-order differential signal is lower than a negative second threshold value and higher than a positive third threshold value is repeated as many times as a value obtained by applying a floor function (n+1)/2.

### Embodiment 5

The emotional change estimating apparatus according to Embodiment 3, wherein the first determination unit determines that the contact condition between the skin and the electrode has changed when a change in which the n-th-order differential signal is higher than a positive fourth threshold value and lower than a negative fifth threshold value is repeated as many times as a value obtained by applying a floor function (n+1)/2.

### Embodiment 6

The emotional change estimating apparatus according to Embodiment 3, 4, or 5, wherein the emotional change estimating unit corrects data on the n-th-order differential signal at a corresponding first period, generates a second signal, and estimates the emotional change based on the second signal when the first determination unit determines that the contact condition has changed.

### Embodiment 7

The emotional change estimating apparatus according to Embodiment 6, wherein the emotional change estimating unit corrects the data on the n-th-order differential signal at the first period based on the n-th-order differential signal at a period other than the first period.

### Embodiment 8

The emotional change estimating apparatus according to Embodiment 2, wherein the emotional change estimating unit changes the first threshold value according to the first signal.

### Embodiment 9

The emotional change estimating apparatus according to Embodiment 8, wherein the emotional change estimating unit changes the first threshold value so that the first threshold value shall be a monotonic function of an average value of the first signal.

### Embodiment 10

The emotional change estimating apparatus according to Embodiment 1 or 2, wherein

the differential signal output unit outputs an m-th-order differential signal (where m is an integer larger than n) of the first signal, and

the emotional change estimating apparatus further includes a second determination unit for determining whether the first signal contains a noise signal caused by a change in contact condition between the electrode and the skin based on the n-th-order differential signal and the m-th-order differential signal.

### Embodiment 11

The emotional change estimating apparatus according to Embodiment 10, wherein the second determination unit determines that the noise signal is contained when a ratio of the amplitude of the m-th-order differential signal to the amplitude of the n-th-order differential signal is larger than a sixth threshold value, and the second determination unit determines that the noise signal is not contained when the ratio of the amplitude of the m-th-order differential signal to the amplitude of the n-th-order differential signal is smaller than a seventh threshold value.

### Embodiment 12

The emotional change estimating apparatus according to Embodiment 10 or 11, wherein the emotional change estimating unit corrects data on the n-th-order differential signal at a corresponding second period, generates a third signal, and estimates the emotional change based on the third signal when the second determination unit determines that the noise signal is contained.

### Embodiment 13

The emotional change estimating apparatus according to Embodiment 12, wherein the emotional change estimating unit corrects the data on the n-th-order differential signal at the second period based on the n-th-order differential signal at a period other than the second period.

### Embodiment 14

The emotional change estimating apparatus according to Embodiment 1 or 2, further including:

a first determination unit for determining whether the contact condition between the skin and the electrode has changed based on the n-th-order differential signal; and

a second determination unit for determining whether the first signal contains a noise signal caused by a change in contact condition between the electrode and the skin based on the n-th-order differential signal and the m-th-order differential signal.

### Embodiment 15

The emotional change estimating apparatus according to Embodiment 14, wherein the emotional change is detected by a capacitance measurement in contact with the skin of the living body.

### Embodiment 16

The emotional change estimating apparatus according to any one of Embodiments 1 to 15, wherein the first signal and the n-th-order differential signal are discrete time signals.

### Embodiment 17

The emotional change estimating apparatus according to any one of Embodiments 1 to 16, wherein the n-th-order differential signal is a signal obtained from data adjacent in terms of time in the first signal.

### Embodiment 18

The emotional change estimating apparatus according to any one of Embodiments 1 to 17, wherein the integer in the n-th-order differential signal is n=1.

### Embodiment 19

The emotional change estimating apparatus according to Embodiment 18, wherein the integer in the m-th-order differential signal is m=2.

### Embodiment 20

An emotional change estimating method including:

measuring an electrophysical quantity on at least one electrode adapted to come into contact with the skin of a living body, and outputting a first signal corresponding to the electrophysical quantity;

receiving input of data on the first signal at multiple times, and outputting an n-th-order differential signal (where n is an integer of 1 or more) of the first signal; and

estimating an emotional change of the living body based on the n-th-order differential signal.

### Embodiment 21

The emotional change estimating method according to Embodiment 20, wherein estimating the emotional change is to compare the n-th-order differential signal with a first threshold value in order to estimate the emotional change of the living body.

### Embodiment 22

The emotional change estimating method according to Embodiment 20 or 21, further including determining whether a contact condition between the skin and the electrode has changed based on the n-th-order differential signal.

### Embodiment 23

The emotional change estimating method according to Embodiment 22, wherein determining whether the contact condition has changed is to determine that the contact condition between the skin and the electrode has changed when a change in which the n-th-order differential signal is lower than a negative second threshold value and higher than a positive third threshold value is repeated as many times as a value obtained by applying a floor function (n+1)/2.

### Embodiment 24

The emotional change estimating method according to Embodiment 22, wherein determining whether the contact condition has changed is to determine that the contact condition between the skin and the electrode has changed when a change in which the n-th-order differential signal is higher than a positive fourth threshold value and lower than a negative fifth threshold value is repeated as many times as a value obtained by applying a floor function (n+1)/2.

### Embodiment 25

The emotional change estimating method according to Embodiment 22, 23, or 24, wherein estimating the emotional change is to correct data on the n-th-order differential signal at a corresponding first period, generate a second signal, and estimate the emotional change based on the second signal when it is determined that the contact condition has changed by determining whether the contact condition has changed.

### Embodiment 26

The emotional change estimating method according to Embodiment 25, wherein estimating the emotional change is to correct the data on the n-th-order differential signal at the first period based on the n-th-order differential signal at a period other than the first period in order to estimate the emotional change.

### Embodiment 27

The emotional change estimating method according to Embodiment 21, wherein estimating the emotional change is to change the first threshold value according to the first signal in order to estimate the emotional change.

### Embodiment 28

The emotional change estimating method according to Embodiment 27, wherein estimating the emotional change is to change the first threshold value so that the first threshold value shall be a monotonic function of an average value of the first signal in order to estimate the emotional change.

### Embodiment 29

The emotional change estimating method according to Embodiment 20 or 21, wherein

outputting the n-th-order differential signal includes outputting an m-th-order differential signal (where m is an integer larger than n) of the first signal, and

the emotional change estimating method further includes determining whether the first signal contains a noise signal caused by a change in contact condition between the electrode and the skin based on the n-th-order differential signal and the m-th-order differential signal.

### Embodiment 30

The emotional change estimating method according to Embodiment 29, wherein determining whether the noise signal is contained is to determine that the noise signal is contained when a ratio of the amplitude of the m-th-order differential signal to the amplitude of the n-th-order differential signal is larger than a sixth threshold value, and that the noise signal is not contained when the ratio of the amplitude of the m-th-order differential signal to the amplitude of the n-th-order differential signal is smaller than a seventh threshold value.

### Embodiment 31

The emotional change estimating method according to Embodiment 29 or 30, wherein estimating the emotional change is to correct data on the n-th-order differential signal at a corresponding second period, generate a third signal, and estimate the emotional change based on the third signal when it is determined that the noise signal is contained by determining whether the noise signal is contained.

### Embodiment 32

The emotional change estimating method according to Embodiment 31, wherein estimating the emotional change is to correct the data on the n-th-order differential signal at the second period based on the n-th-order differential signal at a period other than the second period in order to estimate the emotional change.

### Embodiment 33

The emotional change estimating method according to Embodiment 20 or 21, further including:

determining whether the contact condition between the skin and the electrode has changed based on the n-th-order differential signal; and

determining whether the first signal contains a noise signal caused by a change in contact condition between the electrode and the skin based on the n-th-order differential signal and the m-th-order differential signal.

### Embodiment 34

An emotional change estimating method including:

measuring an electrophysical quantity on at least one electrode adapted to come into contact with the skin of a living body, and outputting a first signal corresponding to the electrophysical quantity;

inputting the first signal at multiple times, acquiring an n-th-order differential signal (where n is an integer of 1 or more) of the first signal and acquiring an m-th-order differential signal (where m is an integer larger than n) of the first signal;

determining whether a contact condition between the skin and the electrode has changed based on the n-th-order differential signal, and determining whether the first signal contains a noise signal caused by a change in contact condition between the electrode and the skin based on the n-th-order differential signal and the m-th-order differential signal;

estimating an emotional change of the living body based on the n-th-order differential signal from the differential signal output step; and

correcting the n-th-order differential signal in estimating the emotional change when a contact failure occurs.

### Embodiment 35

A program for causing a computer to execute each step according to any one of Embodiments 20 to 34.

### Embodiment 36

A computer-readable recording medium on which the program for executing each step according to Embodiment 35 is recorded.

### REFERENCE SIGNS LIST

1, 1a, 1b electrode
10, 20, 30, 40, 50 emotional change estimating apparatus
11 current source
12 electrophysical quantity measuring unit
12a amplifier (I/V conversion)
12b A/D converter
13 data accumulation unit
14 differential signal output unit
14a n-th-order differential signal output unit
14b m-th-order differential signal output unit
15 emotional change estimating unit
16 output unit
60 vital signal measuring apparatus
62 vital signal measuring unit
62a, 72a amplifier
62b, 72b A/D converter
63, 73 processing unit
64, 74 time differentiation signal output unit
64a, 74a n-th-order differential signal output unit
65, 75 output unit
70 plethysmogram measuring apparatus
71a light-emitting element
71b light-receiving element
75 driver
72 plethysmogram measuring unit
81 arm band
82 fixing tool

1. A vital signal measuring apparatus attached to a living body to measure a vital signal of the living body, comprising:
a vital signal measuring unit for measuring the vital signal and outputting a first signal corresponding to the vital signal;
a time differentiation signal output unit for receiving the first signal, and outputting an n-th-order time differentiation signal of the first signal, where n is an integer of 1 or more; and
a contact condition determination unit for determining a contact condition between the vital signal measuring apparatus and the living body based on the n-th-order time differentiation signal.

2. The vital signal measuring apparatus according to claim 1, further comprising an emotional change estimating unit for estimating an emotional change of the living body based on the n-th-order time differentiation signal.

3. The vital signal measuring apparatus according to claim 2, wherein the emotional change estimating unit compares the n-th-order time differentiation signal with a first threshold value to estimate the emotional change of the living body.

4. The vital signal measuring apparatus according to claim 2, wherein n-th-the contact condition determination unit determines that the contact condition between the vital signal measuring apparatus and the living body has changed based on the n-th-order time differentiation signal and a negative threshold value.

5. The vital signal measuring apparatus according to claim 2, wherein the contact condition determination unit determines that the contact condition between the vital signal measuring apparatus and the living body has changed when a change in which the n-th-order time differentiation signal is lower than a negative second threshold value and higher than a positive third threshold value is repeated as many times as a value obtained by applying a floor function (n+1)/2.

6. The vital signal measuring apparatus according to claim 2, wherein the contact condition determination unit determines that the contact condition between the vital signal measuring apparatus and the living body has changed when a change in which the n-th-order time differentiation signal is higher than a positive fourth threshold value and lower than a negative fifth threshold value is repeated as many times as a value obtained by applying a floor function (n+1)/2.

7. The vital signal measuring apparatus according to claim 2, wherein the emotional change estimating unit corrects data on the n-th-order time differentiation signal at a corresponding first period, generates a second signal, and estimates the emotional change based on the second signal when the contact condition determination unit determines that the contact condition has changed.

8. The vital signal measuring apparatus according to claim 7, wherein the emotional change estimating unit corrects the data on the n-th-order time differentiation signal at the first period based on the n-th-order time differentiation signal at a period other than the first period.

9. The vital signal measuring apparatus according to claim 3, wherein the emotional change estimating unit changes the first threshold value according to the first signal.

10. The vital signal measuring apparatus according to claim 9, wherein the emotional change estimating unit changes the first threshold value so that the first threshold value shall be a monotonic function of an average value of the first signal.

11. The vital signal measuring apparatus according to claim 1, wherein the contact condition determination unit determines that the contact condition between the vital signal measuring apparatus and the living body has changed based on the n-th-order time differentiation signal and a negative threshold value.

12. The vital signal measuring apparatus according to claim 1, wherein the contact condition determination unit determines that the contact condition between the vital signal measuring apparatus and the living body has changed when a change in which the n-th-order time differentiation signal is lower than a negative second threshold value and higher than a positive third threshold value is repeated as many times as a value obtained by applying a floor function (n+1)/2.

13. The vital signal measuring apparatus according to claim 1, wherein the contact condition determination unit determines that the contact condition between the vital signal measuring apparatus and the living body has changed when a change in which the n-th-order time differentiation signal is higher than a positive fourth threshold value and lower than a negative fifth threshold value is repeated as many times as a value obtained by applying a floor function (n+1)/2.

14. The vital signal measuring apparatus according to claim 11, further comprising a first data correction unit for correcting data on the first signal at a corresponding first period to generate a second signal when the contact condition determination unit determines that the contact condition has changed.

15. The vital signal measuring apparatus according to claim 14, wherein the first data correction unit corrects the data on the first signal at the first period based on the first signal at a period other than the first period.

16. The vital signal measuring apparatus according to claim 1, wherein
the time differentiation signal output unit outputs an m-th-order time differentiation signal of the first signal, where m is an integer larger than n, and
the vital signal measuring apparatus further comprises a noise signal determination unit for determining whether the first signal contains a noise signal caused by a change in contact condition between the vital signal measuring apparatus and the living body based on the n-th-order time differentiation signal and the m-th-order time differentiation signal.

17. The vital signal measuring apparatus according to claim 16, wherein the noise signal determination unit determines that the noise signal is contained when a ratio of amplitude of the m-th-order time differentiation signal to amplitude of the n-th-order time differentiation signal is larger than a sixth threshold value, and the noise signal determination unit determines that the noise signal is not contained when the ratio of the amplitude of the m-th-order time differentiation signal to the amplitude of the n-th-order time differentiation signal is smaller than a seventh threshold value.

18. The vital signal measuring apparatus according to claim 16, wherein the contact condition determination unit further includes a second data correction unit for correcting data on the first signal at a corresponding second period and generating a third signal when the noise signal determination unit determines that the noise signal is contained.

19. The vital signal measuring apparatus according to claim 18, wherein the second data correction unit corrects the data on the first signal at the second period based on the first signal at a period other than the second period.

20. The vital signal measuring apparatus according to claim 16, further comprising an emotional change estimating unit for estimating an emotional change of the living body based on the n-th-order time differentiation signal,
wherein the emotional change estimating unit corrects data on the n-th-order time differentiation signal at a corresponding second period, generates a third signal, and estimates the emotional change based on the third signal when the noise signal determination unit determines that the noise signal is contained.

21. The vital signal measuring apparatus according to claim 20, wherein the emotional change estimating unit corrects the data on the n-th-order time differentiation signal at the second period based on the n-th-order time differentiation signal at a period other than the second period.

22. The vital signal measuring apparatus according to claim 1, wherein the first signal and the n-th-order time differentiation signal are discrete time signals.

23. The vital signal measuring apparatus according to claim 1, wherein the vital signal measuring unit is an electrophysical quantity measuring unit for measuring an electrophysical quantity on at least one electrode adapted to come into contact with a skin of the living body, and outputting a signal corresponding to the electrophysical quantity as the first signal.

24. The vital signal measuring apparatus according to any to claim 1, wherein the vital signal measuring unit measures a pulse wave of the living body from a plethysmogram sensor, and outputs a signal corresponding to the pulse wave as the first signal.

25. A method for estimating a contact condition, which determines a contact condition of a vital signal measuring apparatus for measuring a vital signal in contact with a living body, comprising:
measuring the vital signal and outputting a first signal corresponding to the vital signal;
outputting an n-th-order time differentiation signal of the first signal, where n is an integer of 1 or more; and
determining n-th-a contact condition between the vital signal measuring apparatus and the living body based on the n-th-order time differentiation signal.

26. A program for causing a computer to execute the method for estimating a contact condition according to claim 25.

27. A computer-readable recording medium on which the program according to claim 26 is recorded.
